(19) United States
(12) Patent Application Publication
(10) Pub. No.: US 2026/0272936 A1
(43) Pub. Date: Sep. 17, 2026

(54) TREATMENT OF CANCER WITH A PARG INHIBITOR

(71) Applicant: 858 Therapeutics, Inc., San Diego, CA (US)

(72) Inventors: James M. VEAL, Solana Beach, CA (US); Jeffrey A. STAFFORD, Rancho Santa Fe, CA (US); Gretchen W. BAIN, San Diego, CA (US); Katherine M. BELL-MCGUINN, Zionsville, IN (US); John P. HOLLERAN, San Diego, CA (US)

(21) Appl. No.: 19/671,940

(22) Filed: May 8, 2026

Related U.S. Application Data
(63) Continuation of application No. PCT/US2024/056671, filed on Nov. 20, 2024.
(60) Provisional application No. 63/601,453, filed on Nov. 21, 2023.

(51) Int. Cl. A61K 31/519; A61K 31/17; A61K 31/423; A61K 31/455; A61K 31/495; A61K 33/243; A61K 38/43; A61P 35/04

(52) U.S. Cl. CPC A61K 31/519; A61K 31/17; A61K 31/423; A61K 31/455; A61K 31/495; A61K 33/243; A61K 38/43; A61P 35/04

(57) ABSTRACT

Provided herein are compositions and methods for the treatment of a cancer. Said compositions comprise a PARG inhibitor. Some embodiments comprise combination therapy featuring the PARG inhibitor with at least one additional oncology therapeutic agent.

### CROSS-REFERENCE TO RELATED APPLICATION

This application is a continuation of International Application No. PCT/US2024/056671, filed Nov. 20, 2024, which claims the benefit of U.S. Patent Application No. 63/601,453, filed on Nov. 21, 2023, each of which is hereby incorporated by reference in its entirety.

### BACKGROUND OF THE INVENTION

Poly(ADP-ribose)glycohydrolase (PARG) is an enzyme that plays a critical role in DNA damage response. PARG inhibition or depletion has been shown to be beneficial for the treatment of certain cancer types, and PARG-depleted or inhibited cancer cells also show an increased sensitivity to other therapies such as DNA damaging agents, cell cycle checkpoint inhibitors, and inhibitors of enzymes involved in nucleotide metabolism. PARG inhibitors are anticipated to have utility as a cancer treatment both as single agents and in combination with therapeutic agents and radiotherapy.

### BRIEF SUMMARY

One embodiment provides a method of treating a cancer in a patient in need thereof, comprising administering to the patient (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof.

One embodiment provides a method of treating a cancer in a patient in need thereof, comprising administering to the patient a pharmaceutical composition comprising (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof, and at least one pharmaceutically acceptable excipient.

One embodiment provides a method of treating a cancer in a patient in need thereof, comprising administering to the patient:
(a) a composition comprising (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof; and
(b) at least one oncology therapeutic selected from a DNA damaging agent, a platinum-based chemotherapy, a DNA damage response inhibitor, cell cycle checkpoint inhibitor, a nucleotide metabolism inhibitor, a nucleotide synthesis inhibitor, an endocrine therapy, a hormonal therapy, a SERM therapy, a SERD therapy, an androgen deprivation therapy, a novel hormone therapy, a taxane, a topoisomerase inhibitor, an anti-VEGF therapeutic, an immune checkpoint inhibitor, a kinase inhibitor, a K-RAS inhibitor, a phosphatidylinositol 3-kinases (PI3K) inhibitor, an antibody drug conjugate (ADC), or a radiopharmaceutical.

### BRIEF DESCRIPTION OF THE DRAWINGS

FIG. 1 shows the average concentration-response curve for inhibition of PARG activity by Compound 1.

FIG. 2 shows average concentration-response curves for induction of PAR accumulation by Compound 1.

FIG. 3A shows average concentration-response curves for inhibition of proliferation in human cancer cell lines by Compound 1.

FIG. 3B shows average concentration-response curves for normalized ATP content in human ovarian cancer patient-derived organoids after treatment with Compound 1.

FIGS. 4A-4B shows mean tumor volumes and % body weight changes after treatment with Compound 1 in a HCC1428 breast cancer xenograft model (Study 1).

FIGS. 4C-4D shows mean tumor volumes and % body weight changes after treatment with Compound 1 in a HCC1428 breast cancer xenograft model (Study 2).

FIG. 4E shows % of γH2AX Positive Cells in Tumor Tissue after treatment with Compound 1 in a HCC1428 breast cancer xenograft model (Study 2).

FIGS. 4F-4G show mean tumor volumes and % body weight changes after treatment with Compound 1 in a HBCx-22 breast cancer patient-derived xenograft model.

FIGS. 4H-4I show mean tumor volumes and % body weight changes after treatment with Compound 1 in a HBCx-34 breast cancer patient-derived xenograft model.

FIGS. 5A-5B show mean tumor volumes and % body weight changes after treatment with Compound 1 in a RMUG-S ovarian cancer xenograft model (Study 1).

FIGS. 5C-5F show mean tumor volumes and % body weight changes after treatment with Compound 1 in a RMUG-S ovarian cancer xenograft model (Study 2).

FIGS. 5G-5H show mean tumor volumes and % body weight changes after treatment with Compound 1 in a T025OV ovarian cancer patient-derived xenograft model.

FIG. 6 shows average concentration-response curves for inhibition of proliferation of parental and PARP inhibitor resistant SNU601 gastric cancer cell lines by Compound 1 and the PARP inhibitor niraparib.

FIG. 7 shows concentration-response curves for inhibition of proliferation in the OVCAR3 ovarian cancer cell line by Compound 1 in combination with the ATR inhibitor camonsertib (RP-3500).

FIG. 8A-8C show concentration-response curves for inhibition of proliferation in the HCC1954 breast, HCC1806 breast, and GP2C colorectal cancer cell lines by Compound 1 in combination with the topoisomerase I inhibitor, exatecan.

FIG. 9 shows concentration-response curves for inhibition of proliferation of Patient-derived Ovarian Cancer Models (OCM) by Compound 1 and the PARP inhibitor niraparib

FIGS. 10A-10B shows mean tumor volumes and % body weight changes after treatment with Compound 1 in combination with fulvestrant and the CDK4/6 inhibitor abemaciclib in a HCC1428 breast cancer xenograft model.

FIGS. 11A-11B shows mean tumor volumes and % body weight changes after treatment with Compound 1 in combination with the ATR inhibitor camonsertib in an HCC1806 triple negative breast cancer xenograft model.

### INCORPORATION BY REFERENCE

All publications, patents, and patent applications mentioned in this specification are hereby incorporated by reference for the specific purposes identified herein.

### DETAILED DESCRIPTION

### Certain Terminology

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Pharmaceutically acceptable salt” includes both acid and base addition salts. A pharmaceutically acceptable salt of the heterocyclic PARG inhibitor described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made. The term “treating”, as used herein, unless otherwise indicated, means reversing, alleviating, inhibiting the progress of, or preventing the disorder or condition to which such term applies, or one or more symptoms of such disorder or condition. In some embodiments, the term “treating” includes slowing or delaying the progression of the disease or disorder to which the term is applied. Additionally, in some embodiments, the term “treating” is applied to one or more of the complications resulting from the disease or disorder to which the term is applied. The term “treatment”, as used herein, unless otherwise indicated, refers to the act of treating as “treating” is defined immediately above.

The term “tumor,” or “cancer” as used herein, and unless otherwise specified, refers to a neoplastic cell growth, and includes pre-cancerous and cancerous cells and tissues. Tumors usually present as a lesion or lump. As used herein, “treating” a tumor means that one or more symptoms of the disease, such as the tumor itself, vascularization of the tumor, or other parameters by which the disease is characterized, are reduced, ameliorated, inhibited, placed in a state of remission, or maintained in a state of remission. “Treating” a tumor also means that one or more hallmarks of the tumor may be eliminated, reduced, or prevented by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries.

The term “refractory” or “refractory to therapy” indicates that the patients have never responded to therapy.

The term “relapsed” or “relapsed after therapy” indicates that patients, after initially responding to prior therapy, have progressive disease due to acquired resistance and/or intolerance.

The term “resistance to therapy” or “acquired resistance to therapy” indicates the patients, after initially responding to prior therapy, have progressive disease due to clinical or molecular resistance to the therapy. The acquired resistance can result from emergence of resistant mutations in the molecular target of the therapy, or in the development of physiological functions such as efflux pumps.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The phrase “platins” or “platinum-based chemotherapy” refers to a platinum containing class of chemotherapeutic drug used for treating cancer. Examples of platinum-based chemotherapies include, but are not limited to, cisplatin, carboplatin, satraplatin, heptaplatin, picoplatin, nedaplatin, triplatin, lipoplatin, and oxaliplatin.

The phrase “PARP inhibitor” refers to an agent that inhibits Poly ADP Ribose Polymerase (PARP) activity, including PARP1 and PARP2. A PARP inhibitor may broadly inhibit the PARP family of enzymes or specifically inhibit one isoform, for example PARP1. Examples of PARP inhibitors include, but are not limited to, niraparib, rucaparib, olaparib, talazoparib, veliparib, and AZD5305.

Other aspects, advantages, and features of the invention will become apparent from the detailed description below.

### Poly(ADP-ribose) Glycohydrolase (PARG) Enzyme Function and Cellular Roles

A hallmark of cancer cells is increased levels of damaged DNA and associated replication stress, which can be defined as the slowing or stalling of replication forks during the DNA replication process. The cellular response to damaged DNA and replication stress is the activation of cell-cycle checkpoints and DNA damage response (DDR) mechanisms to arrest the cell cycle and promote repair of the damaged DNA via mechanisms that include single stranded break repair (SSBR) and base excision repair (BER) pathways. DDR and replication stress response represent cancer-specific vulnerabilities, which can be targeted to induce cancer cell senescence or death.

DNA damage and replication stress responses are regulated by poly ADP ribosylation (PARylation), a transient post-translational modification characterized by the polymerization of ADP-ribose molecules onto nuclear proteins to form poly(ADP-ribose) (PAR) chains. PARylation is catalyzed by the PARP (poly ADP-ribose polymerase) family of proteins via the hydrolysis of NAD+ into nicotinamide and ADP-ribose and the polymerization of the ADP-ribose onto acceptor proteins. PAR chain removal is catalyzed by members of the glycohydrolase family of enzymes of which PARG is the primary PAR glycohydrolase and accounts for approximately 90% of the dePARylation activity in vivo (Koh, 2004). PARG has a critical role in the DNA damage repair cycle and is required to complete the DNA break repair cycle initiated by PARP enzymes.

PARG exists as a single gene with isoforms that reside in the nucleus, mitochondria, and cytosol. In humans, the PARG gene is located on a single chromosome, at the locus 10q11.23-21, but can be subjected to alternative splicing, resulting in different PARG isoforms. Different PARG isoforms can be localized in different subcellular locations and have different degrees of catalytic activity.

In addition to its role in DNA damage response, PARG impacts PAR signaling in splicing, transcriptional, and epigenetic pathways. PARG can prevent the accumulation of cytoplasmic PAR, and also parthanatos, a PAR-mediated type of cell death. PARG functions to maintain stable levels of PAR to protect the cell against parthanatos, which is triggered by the release of the apoptosis-inducing factor (AIF) from the mitochondria to the nucleus. PARG can also have a functional role in telomere maintenance and replication by negatively regulating access to telomeric DNA and reversing ADP-ribosylation of telomeric-specific protein TRF1.

### Inhibition of PARG Function

Suppression of PAR chain hydrolysis via PARG depletion or inhibition leads to defective single-strand and double-strand break repair, reduced kinetics of break repair, and hypersensitivity to DNA damaging agents (Ame, 2009; Fisher, 2007; Gravells, 2017; Gravells, 2018, Kassab, 2020). PARG depletion or inhibition has been shown to inhibit proliferation and arrest cells in the S or G2 phase of the cell cycle and/or induce apoptosis alone or in combination with DNA damaging agents or replication stress inducers. Although PARG and PARP1 work in concert to regulate DNA SSB repair, PARG inhibitors show distinct pharmacology as compared to PARP inhibitors in some cancer cells, suggesting that PARG inhibitors could be used to treat a different subset of tumors as compared to PARP inhibitors. PARG depletion reduces survival of BRCA2-deficient cancer cells. PARG inhibitor sensitivity can also occur in BRCA wild-type, HR-proficient cells, and in PARP inhibitor resistant cells (Coulson-Gilmer, 2021; Pillay, 2019; Houl, 2019; James, 2016). Additionally, depletion of TIMELESS or DNA polymerase β (polβ) showed synthetic lethality with PARG inhibition, but not PARP inhibition, in ovarian cancer cell lines (Pillay, 2019; Pillay, 2021; Ali, 2021).

PARG depletion can sensitize lung, cervical and pancreatic cancer cells to y-irradiation or experimental DNA damaging agents (e.g., hydrogen peroxide, methylmethanesulfonate) (Ame, Fouquerel et al. 2009) (Nakadate, Kodera et al. 2013) (Shirai, Poetsch et al. 2013). Deficiency in PARG does not sensitize to all agents (e.g., gemcitabine, camptothecin), indicating a specificity for PARG function with certain pathways of DNA damage repair and chemo- and radiotherapies (Fujihara, Ogino et al. 2009) (Shirai, Fujimori et al. 2013) (Zhou, Feng et al. 2010) (Zhou, Feng et al. 2011).

The unique efficacy of PARG inhibition in certain cancer types may occur because PARG inhibition appears to exploit specific deficiencies in replication fork machinery in cancer cells under conditions of replication stress (Pillay, 2019, Harrision, 2020). PARG inhibition further slows replication fork progression, increases fork stalling, and increases the number of reversed forks (Houl, 2019; Pillay, 2019; Slade, 2020). In support of the replication catastrophe hypothesis, treatment of sensitive cells with the PARG inhibitor PDD00017273 results in an S-phase dependent accumulation of nuclear RPA protein and induction of pan-nuclear γH2AX expression, hallmarks of replication catastrophe (Pillay, 2019; Toledo, 2013).

Inhibition of PARG activity can also be cytotoxic to sensitive cancer cells via additional mechanisms that include necrosis via cellular NAD+ depletion and parthanatos, a PARP-dependent, PAR-mediated, and caspase-independent form of cell death triggered by the nuclear translocation of apoptosis-inducing factor (AIF) and induction of DNA fragmentation (Feng, 2012; Nagashima, 2020; Zhou, 2011). Depletion of PARG, in contrast to PARP depletion, can lead to a drop in NAD levels, resulting in lung cancer cell death as a result of energy failure (Erdelyi, Bai et al. 2009).

In summary, PARG inhibitors can have uses as a cancer treatment both as single agents and in combination with therapeutic agents and radiotherapy. PARG inhibition leads to antitumor efficacy in cancer types that rely on the mechanisms of SSBR, BER, and protection of stalled replication forks under conditions of replication stress, and these mechanisms represent cancer-specific vulnerabilities. PARG inhibition can be used to target cancer cells having background genetic deficiencies that confer sensitivity to a replication catastrophe mechanism of cell death. PARG inhibition can be effective as a therapy against tumors that are resistant to other treatments such as PARP inhibitors and platinum therapies.

### Prior Art PARG Inhibitors

PARG inhibitors have not been studied to the extent of PARP inhibitors. Clinical resistance to PARP inhibitors has already been described, and therefore there is a need to discover alternative inhibitors targeting the DNA damage response pathways. Previously described PARG inhibitors include bicyclic aryl and heteroaryl compounds as described in WO 2016/092326, WO 2016/097749, WO 2021/055744, WO 2018/237296, WO 2020/023802, WO 2020/205646, WO 2022/138812, WO 2023/057389, WO 2023/057394, WO 2023/154913, WO 2023/165571, WO 2023/175184, WO 2023/175185, WO 2023/183850, WO2023/205914, and WO2023208092. There remains a need to find alternative PARG inhibitors which can target DNA damage response pathways. Such inhibitors would be useful as cancer therapeutics as both single agents and in combination with other therapeutics, including against cancers that do not respond or have become resistant to other therapeutics, such as PARP inhibitors and platinum-based therapeutics.

### Heterocyclic PARG Inhibitor

The heterocyclic PARG inhibitor described herein refers to Compound 1 having the structure below, the Chemical Abstract Number of 3012602-18-2, and the chemical name (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide.

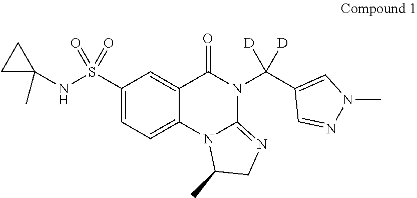 [SMILES: [2H]C([2H])(c1cnn(C)c1)N1C(=O)c2cc(S(=O)(=O)NC3(C)CC3)ccc2N2C1=NC[C@H]2C]

Compound 1 is a small molecule PARG inhibitor. The synthesis and characterization of Compound 1 has been presented in WO 2023/224998 (Application PCT/US2023/022390). Throughout this disclosure when reference is made to a heterocyclic PARG inhibitor, or pharmaceutically acceptable salts or solvates thereof, the reference is to Compound 1, and pharmaceutically acceptable salts or solvates thereof.

### Cancer and Methods of Treatment

One embodiment provides a method of treating a cancer in a patient in need thereof, comprising administering to the patient (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof.

One embodiment provides a method of treating a cancer in a patient in need thereof, comprising administering to the patient a pharmaceutical composition comprising (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof, and at least one pharmaceutically acceptable excipient.

Another embodiment provides the method, wherein the cancer is breast cancer. Another embodiment provides the method, wherein the cancer is a hormone positive breast cancer. Another embodiment provides the method, wherein the cancer is a HER2 negative breast cancer. Another embodiment provides the method, wherein the cancer is a HER2 positive breast cancer. Another embodiment provides the method, wherein the cancer is a triple negative breast cancer. Another embodiment provides the method, wherein the cancer is an invasive ductal carcinoma. Another embodiment provides the method, wherein the cancer is an invasive lobular carcinoma. Another embodiment provides the method, wherein the cancer is a medullary carcinoma. Another embodiment provides the method, wherein the cancer is a metaplastic carcinoma.

Another embodiment provides the method, wherein the cancer is ovarian, fallopian tube, or primary peritoneal cancer. Another embodiment provides the method, wherein the cancer is a mucinous ovarian cancer. Another embodiment provides the method, wherein the cancer is a high-grade serous ovarian cancer. Another embodiment provides the method, wherein the cancer is a clear cell ovarian cancer. Another embodiment provides the method, wherein the cancer is an endometrioid ovarian cancer.

Another embodiment provides the method, wherein the cancer is prostate cancer. Another embodiment provides the method, wherein the cancer is a metastatic castration-resistant prostate cancer (CRPC). Another embodiment provides the method, wherein the cancer is a metastatic castration-sensitive prostate cancer (CSPC). Another embodiment provides the method, wherein the cancer is a high or very high-risk prostate cancer. Another embodiment provides the method, wherein the cancer is a biochemical recurrent prostate cancer with rising PSA.

Another embodiment provides the method, wherein the cancer is gastric, esophageal, or gastro-esophageal junction (GEJ) cancer.

Another embodiment provides the method, wherein the cancer is endometrial or uterine cancer. Another embodiment provides the method, wherein the cancer is a serous endometrial cancer. Another embodiment provides the method, wherein the cancer is an endometrioid endometrial cancer. Another embodiment provides the method, wherein the cancer is a microsatellite stable (MSS) endometrial cancer. Another embodiment provides the method, wherein the cancer is an endometrial cancer with microsatellite instability (MSI).

Another embodiment provides the method, wherein the cancer is cervical cancer.

Another embodiment provides the method, wherein the cancer is lung cancer. Another embodiment provides the method, wherein the cancer is an adenocarcinoma non-small cell lung cancer. Another embodiment provides the method, wherein the cancer is a squamous cell carcinoma non-small cell lung cancer. Another embodiment provides the method, wherein the cancer is a small cell lung cancer.

Another embodiment provides the method, wherein the cancer is pancreatic cancer.

Another embodiment provides the method, wherein the cancer is selected from colorectal, anal, or biliary tract cancer. Another embodiment provides the method, wherein the cancer is a microsatellite stable (MSS) colorectal cancer. Another embodiment provides the method, wherein the cancer is a colorectal cancer with microsatellite instability (MSI).

Another embodiment provides the method, wherein the cancer is skin cancer. Another embodiment provides the method, wherein the cancer is melanoma.

Another embodiment provides the method, wherein the cancer is a cancer associated with the CNS system. Another embodiment provides the method, wherein the cancer is glioblastoma.

Another embodiment provides the method, wherein the cancer is a hematopoietic cancer. Another embodiment provides the method, wherein the cancer is acute myeloid leukemia (AML). Another embodiment provides the method, wherein the cancer is myelodysplastic syndrome (MDS). Another embodiment provides the method, wherein the cancer is diffuse large B cell lymphoma (DLBCL).

Another embodiment provides the method, wherein the cancer is a head and neck cancer.

Another embodiment provides the method, wherein the cancer is thyroid cancer.

Another embodiment provides the method, wherein the cancer is a sarcoma.

Another embodiment provides the method, wherein the cancer is characterized as a having a deficiency in single stranded break repair (SSBR).

Another embodiment provides the method, wherein the cancer is characterized as a having a deficiency in base excision repair (BER).

Another embodiment provides the method, wherein the cancer is characterized as a having a deficiency in protection of the DNA replication fork.

Another embodiment provides the method, wherein the cancer exhibits a mis-regulation in expression of genes selected from BRCA2, BOD1L, DNA-PKC, FEN1, FBH1, POLB, LIG3, PARP1, PCNA, POLK, RAD51, RAD52, RECQ1, RPA, WRN, WRNIP1, XRCC1, or 53BP1.

Another embodiment provides the method, wherein the cancer is characterized as a homologous repair deficient (HRD) cancer.

Another embodiment provides the method, wherein the cancer exhibits a mis-regulation in expression of genes selected from BRCA1, BRCA2, RAD54, RAD51B, ATM, BARDI, CHK1, CHK2, CDK12, RAD51B, RAD54L, RAD51C, RAD51D, PPP22A, BRIPI, CTIP, PALB2, XRCC2, BLM, WRN, or NBS.

Another embodiment provides the method, wherein the cancer exhibits a mis-regulation in expression of genes encoding Fanconi anemia (FA) proteins. Another embodiment provides the method, wherein the cancer exhibits a mis-regulation in expression of genes encoding FA-like genes selected from FANCA, FANCB, FANCC, FANCDI (BRCA2), FANCD2, FANCE, FANCE, FANCG, FANCI, FANJ (BRIPI), FANCL, FANCM, FANCN (RALB2), FANCP (SLX4), FANCS (BRCA1), RAD51C, or XPF.

Another embodiment provides the method, wherein the cancer is characterized by loss of heterozygosity (LOH), telomeric allelic imbalance (TAI), large-scale state transitions (LST), epigenetic events, or BRCA promoter methylation.

Another embodiment provides the method, wherein the cancer is characterized as a having a deficiency in regulation of DNA damage response or cell cycle checkpoints.

Another embodiment provides the method, wherein the cancer exhibits a mis-regulation in expression of genes associated with regulation of DNA damage response or cell cycle checkpoints selected from ATM, ATR, CHK1, CHK2, DNA-PK, PKMYT1, USP1, WEE1, DNA polymerase theta, CDK1, CDK2, CDK4, or CDK6.

Another embodiment provides the method, wherein the cancer is characterized as a having a mis-regulation in expression of a tumor suppressor gene. Another embodiment provides the method, wherein the tumor suppressor gene is selected from TP53 or PTEN1.

Another embodiment provides the method, wherein the cancer is characterized as a having a mis-regulation in an oncogene. Another embodiment provides the method, wherein the oncogene is selected from K-RAS, phosphatidylinositol 3-kinases (PI3K), or BRAF.

Another embodiment provides the method, wherein the cancer is characterized as having amplification of CCNE1.

Another embodiment provides the method, wherein the cancer is metastatic.

Another embodiment provides the method, wherein the method is adjuvant therapy following surgical resection.

Another embodiment provides the method, wherein the method is neo-adjuvant therapy.

Another embodiment provides the method, wherein the patient has relapsed after prior therapy. Another embodiment provides the method, wherein the patient has relapsed after treatment with a platinum-based chemotherapy. Another embodiment provides the method, wherein the patient has relapsed after treatment with a PARP inhibitor.

Another embodiment provides the method, wherein the patient has acquired resistance to prior therapy. Another embodiment provides the method, wherein the patient has acquired resistance to a platinum-based chemotherapy. Another embodiment provides the method, wherein the patient has acquired resistance to treatment with a PARP inhibitor.

Another embodiment provides the method, wherein the patient has acquired resistance to treatment due to a BRCA1 or BRCA2 reversion mutation.

Another embodiment provides the method, wherein the patient is refractory to therapy. Another embodiment provides the method, wherein the patient is refractory to a platinum-based chemotherapy. Another embodiment provides the method, wherein the patient is refractory to treatment with a PARP inhibitor.

One embodiment provides a method of treating a cancer in a patient in need thereof, comprising administering to the patient:
(a) a composition comprising (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof; and
(b) at least one oncology therapeutic selected from a DNA damaging agent, a platinum-based chemotherapy, a DNA damage response inhibitor, cell cycle checkpoint inhibitor, a nucleotide metabolism inhibitor, a nucleotide synthesis inhibitor, an endocrine therapy, a hormonal therapy, a SERM therapy, a SERD therapy, an androgen deprivation therapy, a novel hormone therapy, a taxane, a topoisomerase inhibitor, an anti-VEGF therapeutic, an immune checkpoint inhibitor, a kinase inhibitor, a K-RAS inhibitor, a phosphatidylinositol 3-kinases (PI3K) inhibitor, an antibody drug conjugate (ADC), or a radiopharmaceutical.

Another embodiment provides the method, wherein at least one oncology therapeutic is a DNA damaging agent. Another embodiment provides the method, wherein the DNA damaging agent is selected from hydroxyurea or temozolomide.

Another embodiment provides the method, wherein at least one oncology therapeutic is a platinum-based chemotherapy. Another embodiment provides the method, wherein the platinum-based chemotherapy is selected from cisplatin, carboplatin, satraplatin, heptaplatin, picoplatin, nedaplatin, triplatin, lipoplatin, or oxaliplatin.

Another embodiment provides the method, wherein at least one oncology therapeutic is a DNA damage response inhibitor or a cell cycle checkpoint inhibitor.

Another embodiment provides the method, wherein the DNA damage response inhibitor or cell cycle checkpoint inhibitor is selected from an ATM, ATR, CHK1, CHK2, DNA-PK, PKMYT1, USP1, WEE1, DNA polymerase theta, CDK1, CDK2, CDK4, or CDK6 inhibitor.

Another embodiment provides the method, wherein the DNA damage response inhibitor or cell cycle checkpoint inhibitor is selected from a CDK4 and CDK6 dual inhibitor.

Another embodiment provides the method, wherein the DNA damage response inhibitor or cell cycle checkpoint inhibitor is selected from a KAT6A, KAT6B, or KAT6A/B inhibitor.

Another embodiment provides the method, wherein the DNA damage response inhibitor or cell cycle checkpoint inhibitor is a kinase inhibitor.

Another embodiment provides the method, wherein the DNA damage response inhibitor or cell cycle checkpoint inhibitor is selected from KU-60019, KU-55933, CP-466722, AZD0156, AZD1390, M3541, M4076, XRD-0394, berzosertib, camonsertib, ceralasertib, elimusertib, gartisertib, tuvusertib, IMP9064, ATG-018, ATRN-119, ART0380, dactolisib, rabusertib, prexasertib, AZD7762, BML-277, CHIR-124, CCT245737, GDC-0575, MK-8776, PD0166285, PF-477736, nedisertib, samotolisib, peposertib, AZD7648, CC-115, BR2002, BR101801, LTURM34, LY293646, NU7441, RP-6306, GSK-1520489A, KSQ-4279, ML323, TNG348, ISM3091, adavosertib, azenosertib, Debio0123, IMP7068, PD0166285, SY-4835, ACR-368, ACR-2316, ART558, ART812, ART4215, RP-6685, IDE705, abemaciclib, palbociclib, ribociclib, PF-07104091, BLU-222, INX-315, or ARTS-021. Another embodiment provides the method, wherein the DNA damage response inhibitor or cell cycle checkpoint inhibitor is selected from PF-07248144, OP-3136, or PF-07220060.

Another embodiment provides the method, wherein at least one oncology therapeutic is a nucleotide metabolism inhibitor or a nucleotide synthesis inhibitor. Another embodiment provides the method, wherein the nucleotide metabolism inhibitor or nucleotide synthesis inhibitor is selected from 5-fluorouracil, pemetrexed, leflunomide, brequinar, or methotrexate.

Another embodiment provides the method, wherein at least one oncology therapeutic is an endocrine therapy, a hormonal therapy, a SERM therapy, or a SERD therapy.

Another embodiment provides the method, wherein the endocrine therapy, hormonal therapy, SERM therapy, or SERD therapy is selected from tamoxifen, torimifene, anastrozole, letrozole, exemestane, buserelin, goserelin, leuprorelin, triptorelin, fulvestrant, camizestrant, elacestrant, amcenestrant, giredestrant, imlunestrant, rintodestrant, SHR9549, ZN-c5, D0502, vepdegrestrant, palazestrant, AC682, DT2216, or degarelix.

Another embodiment provides the method, wherein at least one oncology therapeutic is an androgen deprivation therapy or a novel hormone therapy. Another embodiment provides the method, wherein the androgen deprivation therapy or novel hormone therapy is selected from abiraterone, orteronel, bicalutamide, flutamide, enzalutamide, apalutamide, darolutamide, nilutamide, ODM201, AZD3514, BMS641988, ARV-110, ARV-766, CC-94676, AC-0176, HP518, GT20029, or cyproterone acetate.

Another embodiment provides the method, wherein at least one oncology therapeutic is a taxane. Another embodiment provides the method, wherein the taxane is selected from paclitaxel, docetaxel, cabazitaxel, or abraxane.

Another embodiment provides the method, wherein at least one oncology therapeutic is a topoisomerase inhibitor. Another embodiment provides the method, wherein the topoisomerase inhibitor is selected from irinotecan, topotecan, belotecan, doxorubicin, epirubicin, idarubicin, camptothecin, or etoposide.

Another embodiment provides the method, wherein at least one oncology therapeutic is an anti-VEGF therapeutic. Another embodiment provides the method, wherein the anti-VEGF therapeutic is selected from bevacizumab, ramucirumab, aflibercept, sunitinib, pazopanib, regorafenib, or lenvatinib.

Another embodiment provides the method, wherein at least one oncology therapeutic is an immune checkpoint inhibitor. Another embodiment provides the method, wherein the immune checkpoint inhibitor is selected from a CTLA-4 inhibitor, a PD-1 inhibitor, a PD-L1 inhibitor, a bi-specific PD-1/CTLA4 inhibitor, or a bi-specific PD-1/VEGF inhibitor. Another embodiment provides the method, wherein the CTLA-4 inhibitor is selected from ipilimumab or tremelimumab. Another embodiment provides the method, wherein the PD-1 inhibitor is selected from spartalizumab, nivolumab, pembrolizumab, cemiplimab, atezolizumab, avelumab, durvalumab, dostarlimab, retifanlimab, or toripalimab. Another embodiment provides the method, wherein the bi-specific PD-1/CTLA4 inhibitor is selected from AK104, MGD019, XmAb20717, or MEDI5752. Another embodiment provides the method, wherein the bi-specific PD-1/VEGF inhibitor is selected from ivonescimab, LM-299, IMM2510, JS207, AK112, or AI-081.

Another embodiment provides the method, wherein at least one oncology therapeutic is a kinase inhibitor.

Another embodiment provides the method, wherein at least one oncology therapeutic is a K-RAS inhibitor. Another embodiment provides the method, wherein the K-RAS inhibitor is selected from sotorasib, adagrasib, opnurasib, or garsorasib. Another embodiment provides the method, wherein the K-RAS inhibitor is olomorasib.

Another embodiment provides the method, wherein at least one oncology therapeutic is a phosphoinositide 3-kinase inhibitor. Another embodiment provides the method, wherein the phosphoinositide 3-kinase inhibitor is selected from copanlisib, alpelisib, idelalisib, duvelisib, or umbralisib.

Another embodiment provides the method, wherein at least one oncology therapeutic is a BRAF inhibitor. Another embodiment provides the method, wherein the BRAF inhibitor is selected from vemurafenib, dabrafenib or encorafenib.

Another embodiment provides the method, wherein at least one oncology therapeutic is an AKT inhibitor. Another embodiment provides the method, wherein the AKT inhibitor is capivasertib.

Another embodiment provides the method, wherein at least one oncology therapeutic is an antibody drug conjugate. Another embodiment provides the method, wherein the antibody drug conjugate is selected from ado-trastuzumab emtansine, enfortumab vedotin, fam-trastuzumab deruxtecan-nxki, sacituzumab govitecan, cetuximab sarotalocan, disitamab vedotin, tisotumab vedotin, datopotamab deruxtecan, MK-2870, raludotatug deruxtecan, ifinatamab deruxtecan, HS-20089, patritumab deruxtecan, mirvetuximab soravtansine, or farletuzumab ecteribulin. Another embodiment provides the method, wherein the antibody drug conjugate is selected from rinatabart sesutecan, telisotuzumab adizutecan, ABBV-706, YL201, AZD8205, SGN-B7H4V, GSK5764227, or DS-7300.

Another embodiment provides the method, wherein at least one oncology therapeutic is a radiopharmaceutical. Another embodiment provides the method, wherein the radiopharmaceutical is selected from lutetium Lu 177 vipivotide tetraxetan, radium-223 dichloride, sodium iodide I-131, lobenguane iodine-131, lutetium-177, or yttrium-90.

Another embodiment provides the method, wherein the (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof, is administered orally.

Another embodiment provides the method, wherein the oral administration occurs once per day, twice per day, three times per day, every other day, or one to five days per week.

Another embodiment provides the method, wherein the method further comprises administration to the patient of a NAD+ precursor. Another embodiment provides the method, wherein the NAD+ precursor is selected from nicotinic acid, nicotinamide, nicotinamide riboside, or dihydronicotinamide riboside.

### Pharmaceutical Compositions

In certain embodiments, the heterocyclic PARG inhibitor described herein is administered as a pure chemical. In other embodiments, the heterocyclic PARG inhibitor described herein is combined with a pharmaceutically suitable or acceptable carrier (also referred to herein as a pharmaceutically suitable or acceptable excipient, a physiologically suitable or acceptable excipient, or a physiologically suitable or acceptable carrier) selected on the basis of a chosen route of administration and standard pharmaceutical practice.

Provided herein is a pharmaceutical composition comprising the heterocyclic PARG inhibitor as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a method of preparing a pharmaceutical composition comprising mixing the heterocyclic PARG inhibitor as described herein, or a stereoisomer, pharmaceutically acceptable salt, hydrate, or solvate thereof, and a pharmaceutically acceptable carrier.

Provided herein is the method wherein the pharmaceutical composition is administered orally. Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

Provided herein is the method wherein the pharmaceutical composition is administered by injection. In some embodiments, the heterocyclic PARG inhibitor as described herein, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising the heterocyclic PARG inhibitor as described herein, or a stereoisomer, pharmaceutically acceptable salt, hydrate, or solvate thereof, differs depending upon the subject or patient's (e.g., human) condition. In some embodiments, such factors include general health status, age, and other factors. Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

### EXAMPLES

These examples are provided for illustrative purposes only and not to limit the scope of the claims provided herein.

### Example 1: PARG Inhibitory Activity of Compound 1

Compound 1 was evaluated for direct binding to, and inhibition of, recombinant human PARG. Direct binding was determined by surface plasmon resonance (SPR), and inhibition of the enzymatic activity was determined using a PARylated-PARP1 substrate with homogenous time-resolved fluorescence (HTRF) as a readout.

### Methods

Human PARG (amino acids 389-976; Uniprot ID Q86W56-1) containing a N-terminal 6×-Histidine (His) tag followed by a tobacco etch virus (TEV) cleavage site was cloned into the bacterial expression vector pET28a [pET28a-His-TEV-hPARG (389-976)] and transformed into the E. Coli strain BL21 (DE3). BL21 (DE3) cells expressing pET28a-His-TEV-hPARG (389-976) were cultured at 37° C. in Terrific Broth media containing 50 μg/mL kanamycin and 12.5 μg/mL tetracycline. When the optical density was approximately 0.6, the temperature was reduced to 18° C. and IPTG was added to a final concentration of 100 μM and incubation continued at 18° C. for 22 hours. Cells were harvested and lysed by addition of 5 mL/gram cells Buffer A (25 mM Tris, 150 mM NaCl, 5% glycerol, pH 7.5) and incubated on ice for 30 min followed by passing through an ATS high pressure homogenizer (Type: AH110F-25) at 400 bar once, followed by 800 bar twice. The homogenate was centrifuged at 4° C., 8500 rpm for 30 min. The supernatant was incubated with a Ni²⁺ resin for 2 h at 4° C. on a rotator, and then the resin was washed with Buffer A followed by Buffer A containing 20 mM imidazole until no protein signal was observed by SDS-PAGE. Half the protein sample (containing His-tag) was then dialyzed against Buffer C (25 mM Tris, 300 mM NaCl, 5% glycerol, pH 7.5) before concentrating to ~2 mL using ultra-filtration tubes with a molecular weight cut-off of 30 kDa. The concentrated sample was loaded onto a Superdex™ 200 column pre-equilibrated with Buffer B, then washed with Buffer B and fractions collected and analyzed by SDS-PAGE. Protein containing fractions were combined and frozen for use in the SPR assay. The remaining half of the protein was digested with TEV (TEV: protein ratio of 1:10) then dialyzed against Buffer C overnight at 4° C. before purification over a second Ni²⁺ column. The protein (His-tag removed) was loaded onto a Ni²⁺ resin column pre-equilibrated with Buffer C, and then it was washed with Buffer C, Buffer C+10 mM imidazole, Buffer C+20 mM imidazole and Buffer C+250 mM imidazole until no protein signal was observed by SDS-PAGE. The protein sample was concentrated to ~2 mL using ultra-filtration tubes with a molecular weight cut-off of 30 kDa then loaded onto a Superdex™ 200 column pre-equilibrated with Buffer B (25 mM Tris, 150 mM NaCl, 5% glycerol, 1 mM DTT, pH 7.5). The column was washed with Buffer B and fractions collected and analyzed by SDS-PAGE. Protein containing fractions were combined, frozen in liquid N₂ and stored at −80° C. before use in the HTRF assay.

Human PARP (amino acids 2-1014; Uniprot ID P09874-1) containing a N-terminal 6×-His tag followed by a TEV cleavage site was cloned into the insect cell expression vector pFastbac 1. Baculovirus was generated, and High Five insect cells were infected using standard protocols. Infected cells were harvested and lysed by addition of 10 mL/g cells of Buffer A (50 mM Tris, 500 mM NaCl, 5% glycerol, 10 mM β-ME, 100 mM PMSF, 1 protease inhibitor cocktail tablet per 50 mL, pH 8) and sonicated at 150W for 50 times at 4° C. Lysed cells were centrifuged at 13,500 rpm for 30 min at 4° C. and the supernatant collected and centrifuged once more as above. Ni²⁺ resin was pre-equilibrated with Buffer A and then protein loaded on the column. The column was washed with Buffer A+10 mM imidazole followed by Buffer A+20 mM imidazole. Protein fractions were collected by washing with 5 column volumes of Buffer A+250 mM imidazole, and SDS-PAGE was run on each fraction. Pooled fractions containing the protein were loaded onto a Superdex™ 200 column pre-equilibrated with Buffer B (50 mM KH₂PO₄, 150 mM KCl, 5 mM β-ME, 10% glycerol, pH 7.8). The column was washed with Buffer B and fractions collected and analyzed by SDS-PAGE. Fractions containing protein were pooled, aliquoted, frozen with liquid N₂, and stored at −80° C.

Human recombinant His-tagged PARP1 was dialyzed against 1 L of Buffer A (50 mM Tris, 100 mM NaCl, 2 mM DTT, 10 mM MgCl₂, pH 7.5) at 4° C. overnight. The PARylation reaction was initiated by incubating 5.7 μM His-PARP1 protein with 243 μM NAD+ (Millipore Sigma, Burlington, MA, USA, Cat #N0632) and 22 ng/μL of DNase I activated calf thymus DNA (Enzo Life Sciences, Farmingdale, NY, USA, Cat #ALX840040C010) in a 1.45 mL reaction volume for 2 h at room temperature. After the incubation, 250 mM biotinylated-NAD+ (R&D Systems, Minneapolis, MN, USA, Cat #6573/131U) was added to a final concentration of 8.8 μM and incubated at room temperature for an additional 25 min before stopping the reaction by addition of 4.7 μM of the PARP inhibitor A966492 (Selleck Chemicals, Houston, TX, USA, Cat #934162-61-5). The reaction was dialyzed against Storage Buffer (50 mM KH2PO4, pH 7.8, 150 mM KCl, 5 mM β-mercaptoethanol, 10% glycerol) overnight at 4° C. and the final protein concentration measured and snap frozen.

For the SPR assay His-tagged human recombinant PARG (AA 389-976) was coupled to a CM5 biosensor chip via amine coupling. The SPR assay was performed on a Biacore 8K SPR system using Compound 1 serially diluted 2-fold for eight concentrations with a highest starting concentration of 100 nM or 500 nM. The assay was run in 20 mM HEPES, pH 7.0, 150 mM NaCl, 2 mM DTT, 0.001% Tween-20, 5% DMSO at a flow rate of 30 μL/min with a 90 second (Experiment #1) or 60 second (Experiment #2) association time and a 1500 second dissociation time at 25° C.

For the biochemical assay, human recombinant PARG was diluted to 40 nM in cold HTRF buffer (50 mM Tris pH 7.4, 50 mM KCl, 3 mM EDTA, 0.4 mM EGTA, 0.01% Tween-20, 1 mM DTT and 0.1% BSA) and 16 μL added to each well of a 384-well HTRF plate (VWR International, Radnor, PA, Cat #89185-546; Corning #3825). 10% DMSO/90% HTRF buffer alone or varying concentrations of Compound 1 prepared as 10× stocks in 10% DMSO/90% HTRF buffer were then added to the wells and the plate mixed on a plate shaker for 20 seconds. The plate was incubated at 37° C. for 30 minutes then rested at room temperature for 10 minutes. A 10× stock of the PARylated His-tagged PARP1 substrate was prepared by diluting the protein to 100 nMin HTRF buffer at RT, then 2 μL was added to each well before mixing the plate and incubating at RT for 15 minutes to initiate the dePARylation reaction. A 5× stop solution was prepared by adding a PARG inhibitor, anti-His-XL665 antibody (Perkin Elmer, Waltham, MA Cat #61HISXLF) and streptavidin terbium cryptate (Perkin Elmer, Waltham, MA Cat #610SATLA) to a solution of 50 mM Tris, pH 7.4, 0.1% BSA for final concentrations of 200 μM, 1 ng/μL, and 0.02 ng/μL, respectively. The reaction was stopped by the addition of 5 μL of stop solution to each well, mixing on a plate shaker, and incubation for 45 minutes at RT. The plate was read on a Clariostar (BMG Labtech, Cary, NC) plate reader measuring emission values at 665 nm and 620 nm.

Surface plasmon resonance data analysis was performed using Biacore Insight software 3.0. Kinetic binding affinity was calculated from the kinetic association (ka) and dissociation (kd) rates with a 1:1 binding mode. For the biochemical data analysis fluorescence resonance energy transfer (FRET) ratios were calculated using the formula: (emission value 665 nm/emission value 620 nm)*10,000. Maximal PARG activity was determined from the mean FRET ratio of the wells containing PARG+1% DMSO (positive control) and this value was set to 100% activity. Baseline activity was determined from wells containing no PARG+1% DMSO (negative control) and this value was set to 0% activity. The % inhibition of PARG activity of the Compound 1 treated wells relative to the positive and negative controls was graphed and the 50% inhibition concentration (IC₅₀) values generated by non-linear regression analysis using CDD Vault (Collaborative Drug Discovery, Inc., San Diego, CA, USA).

The inhibitory potential of Compound 1 was tested against 38 diverse non-kinase enzymes and 55 receptors, ion channels and neurotransmitter transporters in radioligand competition binding assays using mostly human recombinant proteins. Compound 1 was tested at 10 μM, and a follow-on study determined the IC₅₀ if >50% inhibition was observed in the initial assay. Compound 1 was also tested against an additional panel of 468 protein kinases. Studies were performed at Eurofins (Luxembourg City, Luxembourg) using their published assay formats.

TABLE 1

| | | | | Rmax | |
|---|---|---|---|---|---|
| | K_{D}^{a}/ | | | (Relative | Theoretical |
| METHOD | IC₅₀^{b} (nM) | SEM | n | Units) | Rmax |
| SPR | 1.1^{a} | NA | 2 | 15.9 | 19.8 |
| HTRF | 85^{b} | 0.36 | 14 | NA | NA |

NA = not applicable; n = number of tests

### Results and Conclusion

The binding affinity of Compound 1 for the catalytic domain of human recombinant PARG was evaluated by SPR in single cycle kinetic mode. The kinetic K_{D} values are shown in Table 1. Also shown in Table 1 is the relative amount of PARG bound by Compound 1 (Rₘₐₓ) as compared to the total amount of PARG immobilized on the biosensor chip (Theoretical Rₘₐₓ). Compound 1 showed high affinity binding to the catalytic domain of human PARG with a measured mean K_{D} value of 1.1 nM (n=2).

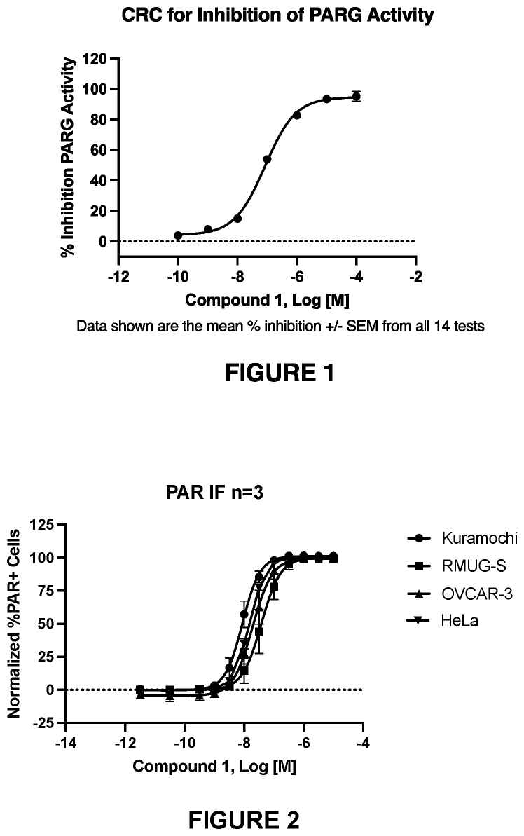

The effect of Compound 1 on inhibition of PARG catalytic activity was evaluated by HTRF using PARylated PARP1 as a substrate. The average concentration-response curve for inhibition of PARG activity is shown in FIG. 1, and the geometric mean IC₅₀ value, standard deviation, and number of tests are presented in Table 1. As shown, Compound 1 inhibited the catalytic activity of PARG in a concentration-dependent manner with a geometric mean IC₅₀ value of 85 nM (n=14).

Compound 1 was screened at 10 UM against a series of 38 diverse non-kinase enzymes and 55 receptors, ion channels, and neurotransmitter transporters and also against an additional panel of 468 protein kinases. Inhibition greater than 50% was observed for only the human A1 receptor (67% inhibition) and the human 5-HT_{2A} receptor (64% inhibition). The follow-on study determined the IC₅₀ values for both the agonist and antagonist binding of Compound 1 to A1 and 5-HT_{2A} receptors. Compound 1 was active only as an antagonist for the 5-HT_{2A} receptor and showed an IC₅₀ value of 21 μM with a dissociation constant (K_{B}) value of 4.2 μM. In the kinase assay panel, Compound 1 at a 10 μM concentration showed 51% inhibition of DRYK1B and <50% inhibition of all other kinases tested.

Compound 1 binds with high affinity to the catalytic domain of human PARG, which leads to potent inhibition of its glycohydrolase activity. Compound 1 is a selective inhibitor of PARG.

### Example 2: Cellular Inhibition of PARG by Compound 1

Inhibition of PARG activity can lead to DNA-damage-induced accumulation of cellular poly(ADP-ribose) (PAR) chains that can be quantitated by immunofluorescence using an anti-PAR antibody. In the present study, Compound 1 was evaluated for the ability to promote DNA damage-induced nuclear PAR accumulation in various cancer cell lines, including the ovarian cancer cell lines, Kuramochi, RMUG-S, and OVCAR-3 and the cervical cancer cell line, HeLa.

### Methods

The HeLa (Cat #CCL-2) and OVCAR-3 (Cat #HTB-161) human cancer cell lines were obtained from ATCC (Manassas, Virginia, USA) and the RMUG-S(Cat #IFO50320) and Kuramochi (Cat #JCRB0098) cell lines were obtained from the National Institutes of Biomedical Innovation, Health and Nutrition's Japanese Collection of Research Bioresources (Ibaraki City, Osaka, Japan).

All cell lines were grown in an incubator at 37° C. in 5% CO₂. HeLa cells were cultured in Dulbecco's Modified Medium (DMEM)+glutaMAX™ (Thermo Fisher Scientific, Waltham, MA, USA Cat #10566-016) containing 1% penicillin/streptomycin (pen/strep) (Thermo Fisher Scientific Cat #15070-063), and 10% fetal bovine serum (FBS) (Thermo Fisher Scientific Cat #A31605-02). Kuramochi cells were cultured in RPMI medium+glutaMAX™ (Thermo Fisher Scientific Cat #72400-047), 10% FBS, and 1% pen/strep. RMUG-S cells were cultured in Ham's F12 medium+glutaMAX™ (Thermo Fisher Scientific Cat #31765-035) containing 10% FBS, and 1% pen/strep. OVCAR-3 cells were cultured in RPMI medium+glutaMAX™ containing 20% FBS, 1% pen/strep, and 0.01 mg/mL bovine insulin (MilliporeSigma, Burlington, MA, USA Cat #10516-5ML).

Compound 1 EC₅₀ values for accumulation of PAR chains were determined using an immunofluorescence high-content imaging assay of PAR staining. Cells were harvested with trypsin (Thermo Fisher Scientific, Cat #25200-056) and plated in black-walled, clear bottom 96-well Corning Costar 3603 plates (VWR, Radnor, PA, USA Cat #29444-008) at 15,000 cells/well in 100 μL normal growth media. The cells were incubated overnight at 37° C., 5% CO₂. The following day, a 1000× Compound 1 concentration curve was prepared by serial dilution of Compound 1 in 100% DMSO for twelve concentrations with the highest starting final concentration of 10 μM. The 1000× test compound curve was diluted 1:200 into growth media to prepare a 5× concentration curve in 0.5% DMSO/media before addition of 25 μL of compound or 0.5% DMSO/media alone to each well of the plate. The plate was mixed gently on a plate shaker and then preincubated at 37° C., 5% CO₂ for 1 hour. A 2.25× stock of MMS (Sigma Cat #129925-5G) plus Compound 1 in media was prepared by diluting a 100 mg/mL stock of MMS prepared in 100% DMSO into growth media containing 1× Compound 1 compound. To induce DNA damage, 100 μL of the 2.25×MMS+Compound 1 was added to each well for a final concentration of 50 μg/mL of MMS and the plate mixed gently and incubated for an hour at 37° C., 5% CO₂. Following the incubation, media was aspirated from each well and the cells fixed by the addition of 100 μL ice-cold 95% methanol/phosphate buffered saline (PBS) for 15 min at −20° C. Cells were washed once with room-temperature (RT) PBS then permeabilized by adding 100 μL of 0.1% Triton X-100 (G-Biosciences, St. Louis, MO, USA) (Cat #786-513) in PBS and incubating for 20 min at RT. After washing once with PBS, 50 μL/well of anti-PAR antibody (Clone 10H, MilliporeSigma, Cat #AM80) diluted 1:1000 in PBS containing 5% FBS and 0.1% Tween-20 (MilliporeSigma, Cat #P9416) was added to each well and the plates incubated at 4° C. overnight. Following 3 washes with PBS at RT, 100 μL/well of goat anti-mouse antibody conjugated to AlexaFluor 488 (Thermo Fisher Scientific Cat #A32723) diluted 1:500 in PBS containing 5% FBS and 0.1% Tween-20 was added and incubated for 1 h at RT. Following 3 washes with PBS, 100 μL/well of DAPI (Thermo Fisher Scientific, Cat #62248) diluted to 1 μg/mL in PBS was added to each well. Nuclear PAR accumulation was evaluated by imaging the plates on an ImageXpress Pico automated cell imaging system (Molecular Devices, San Jose, CA, USA).

Four fields for each well were acquired at 20× magnification resulting in at least 1,000 cells analyzed per well. Percent PAR positive cells were calculated using CellReporterXpress software (Molecular Devices, San Jose, CA, USA) using nuclei detection by DAPI image segmentation then setting a fluorescence intensity threshold for PAR. The threshold for positive PAR staining within the nuclei was based on wells treated with DMSO+MMS, which are PAR negative due to the rapid hydrolysis of the PAR chains by PARG. Percent PAR+ cells were calculated according to the formula: (number of nuclei above PAR threshold/total number of nuclei)*100. Maximal PAR accumulation was determined from the percent PAR+ cells in the wells containing 10 μM Compound 1+MMS (positive control), and this value was set to 100% normalized PAR+ cells. Baseline PAR accumulation was determined from the percent PAR+ cells in the wells containing 0.1% DMSO+MMS (negative control), and this value was set to 0% normalized PAR+ cells. The normalized percent PAR+ cells of the Compound 1 treated wells, calculated relative to the positive and negative controls, was graphed and the 50% effective concentration (EC₅₀) values generated by non-linear regression analysis using CDD Vault software (Collaborative Drug Discovery, Inc., San Diego, CA, USA) (HeLa data) or GraphPad Prism 10 software (GraphPad Software, Boston, MA, USA) (Kuramochi, RMUG-S, and OVCAR-3 data) by non-linear regression analysis using a least squares fit of the log (inhibitor) vs. response—variable slope (four parameters) curve.

TABLE 2

| | Geometric Mean | | |
|---|---|---|---|
| Cell Line | EC₅₀ (μM) | Geometric SEM | Number of Tests |
| HeLa | 0.014 | 0.32 | 26 |
| Kuramochi | 0.009 | 0.94 | 3 |
| RMUG-S | 0.038 | 1.34 | 3 |
| OVCAR-3 | 0.020 | 1.13 | 3 |

### Results and Conclusions

The effect of Compound 1 on inhibition of PAR chain hydrolysis and subsequent accumulation of PAR after DNA damage induced by MMS was evaluated in both ovarian (Kuramochi, OVCAR-3, RMUG-S) and cervical (HeLa) cancer cell lines using an anti-PAR antibody and immunofluorescence readout. The average concentration-response curves for MMS-induced cellular PAR accumulation in Kuramochi, OVCAR-3, RMUG-S and HeLa cell lines are shown in FIG. 2 and the geometric mean EC₅₀ values are shown in Table 2. Compound 1 displayed potent inhibition of cellular PARG activity as determined by the concentration-dependent accumulation of nuclear PAR after MMS treatment in all cell lines tested with geometric mean EC₅₀ values of 0.009 (n=3), 0.038 (n=3), 0.020 (n=3), and 0.014 (n=26) in Kuramochi, RMUG-S, OVCAR-3, and HeLa cells, respectively.

Compound 1 displayed potent inhibition of cellular PARG activity as determined by the accumulation of nuclear PAR after DNA damage in all four cancer cell lines tested with geometric mean EC₅₀ values that ranged from 0.009-0.038 μM.

### Example 3: Inhibition of Cell Proliferation in Cell Culture by Compound 1

Compound 1 was evaluated for its ability to inhibit the proliferation of two ovarian cancer patient-derived organoids (PDOs) and multiple cancer cell lines representing different cancer lineages.

### Methods

The FU-97 (Cat #JCRB1074) human gastric cancer cell line and the Kuramochi (Cat #JCRB0098) and RMUG-S(Cat #IFO50320) human ovarian cancer cell lines were obtained from the National Institutes of Biomedical Innovation, Health and Nutrition's Japanese Collection of Research Bioresources (JCRB) (Ibaraki City, Osaka, Japan). The HCC1419 (Cat #CRL-2326) and HCC1428 (Cat #CRL-2327) human breast cancer cell lines and the NCI-H1650 (Cat #CRL-5883) and NCI-H1792 (Cat #CRL-5895) non-small cell lung cancer cell lines were obtained from ATCC (Manassas, Virginia, USA). The human gastric cancer cell lines GCIY (Cat #RCB0555) and SNU601 (Cat #CSC-C9686L) were obtained from the Riken Bioresource Research Center (Tsukuba, Ibaraki, Japan) and Creative Bioarray (Shirley, NY, USA), respectively. The T025OV ovarian cancer organoid model was developed from a patient-derived xenograft (PDX) model, which was developed from primary serous ovarian carcinoma cells obtained from Discovery Life Sciences (Huntsville, AL, USA, SN #350627149). T026OV organoids were developed from PDX cells obtained from The Jackson Laboratory (Bar Harbor, ME, USA, Cat #TM00335).

All cell lines were grown in an incubator at 37° C. in 5% CO₂. The Kuramochi and SNU-601 cells were cultured in RPMI+glutaMAX™ (Thermo Fisher Scientific, Waltham, MA, USA Cat #74200120) containing 1% penicillin/streptomycin (pen/strep) (Thermo Fisher Scientific Cat #15070-063), and 10% fetal bovine serum (FBS) (Thermo Fisher Scientific Cat #A31605-02). The HCC1419, HCC1428, NCI-H1650, and NCI-H1792 cells were cultured in ATCC-modified RPMI+glutaMAX™ (Thermo Fisher Scientific Cat #A10491-01) containing 1% pen/strep and 10% FBS. The FU-97 cells were cultured in DMEM+glutaMAX™ (Thermo Fisher Scientific, Waltham, MA, USA Cat #10566-016) containing 1% pen/strep, 10% FBS and 0.01 mg/mL bovine insulin (MilliporeSigma, Burlington, MA, USA Cat #10516-5ML). The GCIY cells were cultured in MEM (Thermo Fisher Scientific Cat #11095-080) containing 1% pen/strep and 15% FBS. The RMUG-S cells were cultured in Ham's F12 medium+glutaMAX™ (Thermo Fisher Scientific Cat #31765-035) containing 10% FBS and 1% pen/strep.

T025OV and T026OV PDOs were cultured in PDO complete media on a thin layer of growth-factor reduced Matrigel (Corning, Glendale, AZ, USA Cat #356231). Complete media was prepared by using Advanced DMEM/F12 (Thermo Fisher Scientific, Cat #12634010) that was supplemented with 10 mM HEPES (Thermo Fisher Scientific, Cat #15630080), 1× GlutaMAX™ (Thermo Fisher Scientific, Cat #35050061), 1× Primocin (InvivoGen, San Diego, CA, USA Cat #ant-pm-1), 1× B27 Supplement (Thermo Fisher Scientific, Cat #17504044), 50% Wnt3 a conditioned media (R&D Systems, Minneapolis, MN, USA, Cat #5036-WN-010), 10% RSPO-1 conditioned media (Peprotech, Cranbury, NJ, USA, Cat #120-38), 100 ng/ml human Noggin (Peprotech, Cat #120-10C), 50 ng/ml human EGF (Peprotech, Cat #AF-100-15), 50 ng/mL human FGF10 (Peprotech, Cat #100-26), 10 μM Y-27632 (SelleckChem, Houston, TX, USA, Cat #S1049), 0.5 M A83-01 (Millipore Sigma, St. Louis, MO, USA, Cat #SML0788), and 0.1% methylcellulose (R&D Systems, Cat #HSC001).

Compound 1 IC₅₀ values for inhibition of cell proliferation were determined by imaging and counting the number of nuclei after compound treatment. Cells were harvested with trypsin (Thermo Fisher Scientific, Cat #25200-056) and plated in black-walled, clear bottom 96-well Corning costar 3603 plates (VWR, Radnor, PA, USA Cat #29444-008) at 7500 cells/well (HCC1428 and HCC1419), 5000 cells/well (Kuramochi, FU-97, SNU-601), 2500 cells/well (RMUG-S, GCIY, NCI-H1650) or 1000 cells/well (NCI-H1792) in 100 μL normal growth media and incubated overnight at 37° C., 5% CO₂. The following day, a 1000× Compound 1 concentration curve was prepared by serial dilution of Compound 1 in 100% DMSO. The 1000× Compound 1 concentration curve was diluted 1:200 into growth media to generate a 5× concentration curve in 0.5% DMSO/growth medium before addition of 25 μL of the 5× compound to the wells. Control wells received 25 μL of 0.5% DMSO/media alone (negative control wells) or 50 μM Compound 1 (positive controls; 10 μM Compound 1 final). The plate was mixed gently on a plate shaker, covered with Breathe-Easy biofilm (Diversified Biotech, Cat #BEM-1), and incubated at 37° C., 5% CO₂ for 6 days. Following the incubation period, a 125 μL fixation/permeabilization solution containing 8% paraformaldehyde (Fisher Scientific, Cat #50-980-495, part of Thermo Fisher Scientific) and 0.2% Triton X-100 (Thermo Fisher Scientific, Cat #327372500) in PBS was added to each well of the plate, and the plates were incubated at room temperature for 20 min. The solution was aspirated and cells washed by the addition of 100 μL PBS. After aspiration of the PBS, nuclei were stained by the addition of 100 μL 1 μg/mL DAPI (Thermo Fisher Cat #62248) in PBS. Nuclei were counted by imaging the plates on an ImageXpress Pico automated cell imaging system (Molecular Devices, San Jose, CA, USA).

Compound 1 IC₅₀ values for inhibition of organoid colony formation were determined by measuring ATP levels after 7 days of treatment utilizing Cell Titer Glo 3D reagent (Promega, Madison, WI, USA Cat #G9683). Assay plates were prepared by adding 25 μL of growth factor reduced Matrigel (Corning, #356231) to each well of a 96-well, white walled, clear bottom tissue culture plate (Corning, Cat #3610). Assay plates were then incubated at 37° C. for 15 minutes to polymerize the gel matrix. T025OV and T026OV dissociated PDO cells were then re-suspended at 1×10⁵ cells/mL in PDO complete media and 100 μL added to each well of the assay plate (10,000 cells/well final) and incubated overnight at 37° C., 5% CO₂. The following day, a 1000× Compound 1 concentration curve was prepared by serial dilution of Compound 1 in 100% DMSO with the highest starting concentration of 50 mM (for a final concentration of 50 μM). The 1000× Compound 1 concentration curve was diluted in PDO base media (Advanced DMEM/F12 containing 10% FBS, 1× Primocin, 1× GlutaMAX™, and 10 mM HEPES) to generate a 20× concentration curve in growth medium before addition of 5 μL of the 20× compound to the wells. Control wells received 5 μL of 2% DMSO/media alone (negative control wells, 0.1% DMSO final) or 20 μM Bortezomib in 2% DMSO/media (positive control wells; 1 μM final). The plate was mixed gently on a plate shaker, covered with Breathe-Easy biofilm (Diversified Biotech, Cat #BEM-1) and incubated at 37° C., 5% CO₂ for 7 days. Following compound incubation, 100 μL of Cell Titer Glo 3D was added in a 1:1 ratio to each assay well. Plates were mixed gently on a plate shaker for 5 minutes, then allowed to equilibrate at room temperature for 25 minutes. Luminescence values (RLU) were read-out using a Clariostar plate reader (BMG Labtech, Cary, NC).

Four fields in each well were acquired using the DAPI channel at 10× magnification resulting in ~25% well coverage. Using high-content software (Cell Reporter Xpress), the nuclei were detected and counted. The percent proliferation after treatment was determined by normalizing the number of nuclei in treated wells to the DMSO control wells (negative controls=100% proliferation) and the 10 μM Compound 1 control wells (positive controls=0% proliferation). Concentration-response-curves and IC₅₀ or IC₉₀ values for percent inhibition of proliferation were generated by non-linear regression analysis using CDD Vault software (Collaborative Drug Discovery, Inc., San Diego, CA, USA) (Kuramochi and RMUG-S) or GraphPad Prism 10 software (GraphPad Software, Boston, MA, USA) (HCC1428, HCC1419, FU-97, GCIY, SNU-601, NCI-H1650, NCI-H1792) by non-linear regression analysis using a least squares fit of the log (inhibitor) vs. response—Variable slope (four parameters) curve.

The percent inhibition of 3D colony growth after compound treatment was evaluated by normalizing the RLU values for ATP content in the Compound 1 treated wells to the DMSO control wells and control wells that were treated with bortezomib, an apoptosis inducing factor that results in 100% organoid cell death. Concentration-response-curves and IC₅₀ values for inhibition of ATP content were generated by non-linear regression analysis using CDD Vault software (Collaborative Drug Discovery, Inc., San Diego, CA, USA).

TABLE 3

| Cell Line/ | | IC₅₀ | IC₅₀ | IC₉₀ | IC₉₀ | |
|---|---|---|---|---|---|---|
| PDO | Cancer Lineage | (μM) | SEM | (μM) | SEM | n |
| Kuramochi | High Grade Serous | 0.046 | 0.43 | 0.133 | 0.39 | 13 |
| | Ovarian Cancer | | | | | |
| RMUG-S | Ovarian Mucinous | 0.075 | 0.37 | 0.208 | 0.37 | 13 |
| | Cystadenocarcinoma | | | | | |
| HCC1419 | ER+/HER2+ Breast | 0.050 | 0.82 | 0.206 | 1.32 | 3 |
| | Carcinoma | | | | | |
| HCC1428 | ER+/HER2− Breast | 0.131 | 0.77 | 0.363 | 0.82 | 3 |
| | Carcinoma | | | | | |
| FU-97 | Gastric | 0.178 | 1.03 | 0.833 | 0.96 | 6 |
| | Adenocarcinoma | | | | | |
| GCIY | Gastric | 0.028 | 0.83 | 0.125 | 0.98 | 4 |
| | Adenocarcinoma | | | | | |
| SNU-601 | Gastric | 0.149 | 0.59 | 0.539 | 0.57 | 4 |
| | Adenocarcinoma | | | | | |
| NCI-H1650 | Lung | 0.100 | 1.14 | 0.320 | 1.06 | 3 |
| | Adenocarcinoma | | | | | |
| NCI-H1792 | Lung | 1.150 | 1.30 | 4.280 | 1.11 | 3 |
| | Adenocarcinoma | | | | | |
| T025OV | Ovarian Cancer | 1.070 | 0.70 | 10.692 | 0.77 | 7 |
| | PDO | | | | | |
| T026OV | Ovarian Cancer | 0.213 | 1.84 | 0.567 | 1.27 | 3 |
| | PDO | | | | | |

SEM = standard of the mean;
IC50 and IC90 values are geometric means;
n = number of tests;
PDO = patient derived organoid

### Results and Conclusions

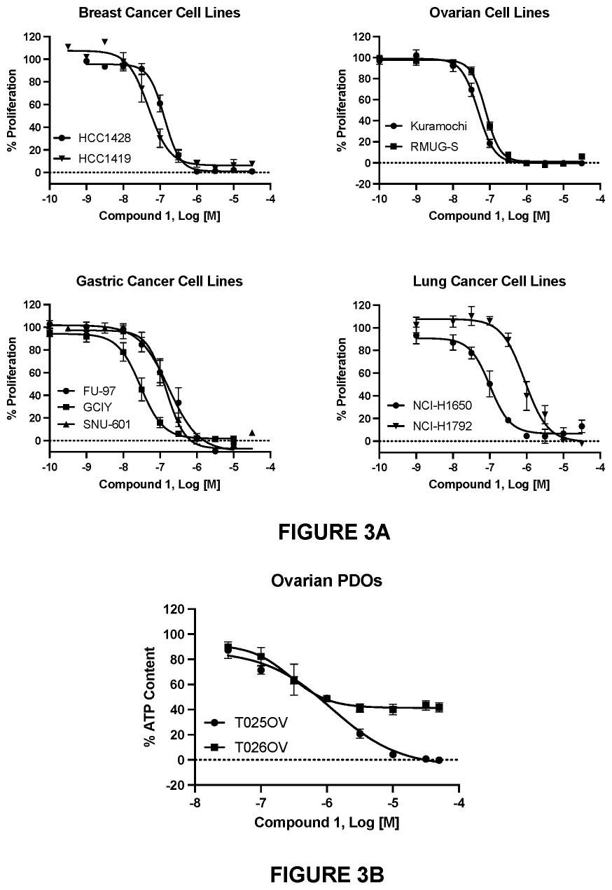

The potency of Compound 1 for inhibiting cell proliferation in breast (HCC1428, HCC1419), ovarian (RMUG-S, Kuramochi), gastric (FU-97, GCIY, SNU-601), and lung (NCI-H1650, NCI-H1792) cancer cell lines was evaluated by nuclei counting after six days of treatment. The average concentration-response curves for percent proliferation are shown in FIG. 3A, and the geometric mean IC₅₀ values for inhibition of proliferation are shown in Table 3. Compound 1 displayed potent and concentration-dependent inhibition of proliferation of cancer cell lines representing a number of distinct lineages. In the ER+/HER2+HCC1419 and ER+/HER2-HCC1428 breast cancer cell lines, Compound 1 inhibited cell growth with mean IC₅₀/IC₉₀ values of 0.050/0.206 (n=3) and 0.131/0.363 μM (n=3), respectively. In the Kuramochi high-grade serous ovarian cancer and RMUG-S ovarian mucinous cystadenocarcinoma lines, Compound 1 inhibited cell growth with mean IC₅₀/IC₉₀ values of 0.046/0.133 (n=13) and 0.075/0.208 UM (n=13), respectively. In the FU-97, GCIY, and SNU-601 gastric adenocarcinoma cell lines, Compound 1 inhibited cell growth with mean IC₅₀/IC₉₀ values of 0.178/0.833 (n=6), 0.028/0.125 (n=4), and 0.149/0.539 μM (n=4), respectively. In the NCI-H1650 and NCI-H1792 non-small cell lung adenocarcinoma cell lines, Compound 1 inhibited cell growth with mean IC₅₀/IC₉₀ values of 0.100/0.320 μM (n=3) and 1.150/4.280 μM (n=3), respectively.

The potency of Compound 1 for inhibiting colony formation in two ovarian cancer PDOs was evaluated via measurement of ATP content in the organoids after seven days of treatment and normalization to DMSO and bortezomib treated wells. The average concentration-response curves for percent ATP content are shown in FIG. 3B, and the geometric mean IC₅₀ and IC₉₀ values for inhibition of ATP content are shown in Table 3. Compound 1 displayed potent and concentration-dependent reductions in cellular ATP content in the T025OV serous carcinoma and T026OV papillary serous adenocarcinoma ovarian cancer PDOs with geometric mean IC₅₀/IC₉₀ values of 1.070/10.692 (n=7) and 0.213/0.567 (n=3), respectively. The T026OV PDO appeared more sensitive to Compound 1 based on mean IC₅₀ value, however, T026OV displayed a maximum reduction in cellular ATP content of approximately 50%. This phenotype is indicative of cell growth inhibition, but not induction of cell death. Compound 1 treatment of T025OV cells reduced cellular ATP content to the level of the bortezomib control, which is consistent with an induction of cell death and complete loss of cells.

Compound 1 showed potent and concentration-dependent inhibition of proliferation of breast, ovarian, gastric, and lung cancer cell lines with geometric mean IC₅₀ values that ranged from 0.028-1.15 μM and geometric mean IC₉₀ values that ranged from 0.125-4.280 μM. Additionally, Compound 1 showed potent, concentration-dependent inhibition of proliferation of two ovarian cancer PDOs, representing serous carcinoma and papillary serous adenocarcinoma, with geometric mean IC₅₀ values of 0.213 and 1.07 UM and respective geometric mean IC₉₀ values of 0.567 and 10.692 μM. The phenotypic response to Compound 1 treatment varied between the two organoid models; Compound 1 treatment reduced ATP content by approximately 50% in the T026OV papillary serous adenocarcinoma cells versus approximately 100% in the T025OV serous carcinoma cells.

### Example 4A: Efficacy of Compound 1 in a Breast Cancer CDX Model

The anti-tumor activity and tolerability of Compound 1 was evaluated in a cell-line derived mouse xenograft model (CDX) using the ER+HER2-BRCA2 mutant HCC1428 human breast cancer cell line. The HCC1428 cells are a cisplatin-resistant BRCA2-mutated breast cancer cell line that contains a secondary genetic change in BRCA2 that rescues BRCA2 homologous recombination repair function (Sakai, 2008, Nature).

### Methods

Two separate studies were performed. The CDX model was established by subcutaneous inoculation of HCC1428 cells into the right flank of BALB/c nude mice. Mice were injected subcutaneously with estradiol benzoate one day prior to cell inoculation and then twice a week thereafter to promote optimal growth of the ER+ cell line. Groups of 8 animals each were established by random assignment. Tumor volumes and body weights were measured twice weekly. The tumor volume (TV) in mm³ was estimated using the formula: TV=a×b²/2, where a and b are the long and short diameters of a tumor, respectively. For each treatment group, if the mean TV at the end of study was greater than the mean tumor volume at treatment initiation, then tumor growth inhibition (TGI) was calculated using the formula: % TGI=[1-(Mean TreatedTV_{Final}−Mean Treated TV_{Initial})/(Mean Vehicle TVFinal−Mean VehicleTV_{Initial})]×100. If the mean TV of the treatment group at the end of study was less than the mean tumor volume at treatment initiation, then tumor regression (REG) was calculated using the formula: % REG=[(Mean Treated TV_{Initial}−Mean Treated TV_{Final})/Mean TreatedTVInitial]×100. Statistical analysis of end-of-study tumor volumes was performed using two-way repeated measures ANOVA followed by post hoc comparisons of the mean.

In the first study (Study 1), Compound 1 treatment was initiated when the mean tumor volume of each group was 177 mm³. Compound 1 was administered to mice orally for 25 days either once daily (QD) at a dose of 100 mg/kg or twice daily (BID) at a dose of 75 mg/kg based on body weight. The PARP inhibitor niraparib was included as a comparator and dosed orally QD at a dose of 45 mg/kg.

In the second study (Study 2), Compound 1 treatment was initiated when the mean tumor volume of each group was 178 mm³. Compound 1 was administered to mice orally for 25 days either once daily (QD) at dose levels of 10, 30, 50 or 100 mg/kg or twice daily (BID) at dose levels of 50 or 70 mg/kg. The mice in the 50 mg/kg BID group were dosed every day whereas the mice in the 70 mg/kg group were dosed for the first 5 days of each 7-day cycle, such that the total weekly dose was the same between these two groups. At the end of the study, tumor tissue was collected for the vehicle, 50 mg/kg BID, and 30, 50 and 100 mg/kg QD dose groups and formalin-fixed and paraffin embedded (FFPE) for γH2AX immunohistochemistry. γH2AX expression in FFPE tumor tissue was evaluated by staining and was performed using immunohistochemistry and validated antibody conditions at Histowiz (Long Island City, NY, USA). The γH2AX IHC images were then quantified in-house using QuPath software. The total number of nuclei objects per tumor section based on hematoxylin counter-staining (blue) were segmented using a Stardist algorithm and the number of γH2AX positive DAB (3,3′ Diaminobenzidine) stained cells (brown) quantified using object classification with a pre-trained random forest model based on DAB staining parameters including intensity and standard deviation.

% γH2AX positive=(#γH2AX positive nuclei/#total nuclei)*100

Statistical analysis of the γH2AX staining was conducted using Ordinary one-way ANOVA with Dunnett's multiple comparisons test and differences considered significant at a calculated probability p≤0.05.

TABLE 4A

| | Compound 1 | | % TGI or | |
|---|---|---|---|---|
| Study | Dose (mg/kg) | Treatment Regimen | REG | P-value |
| 1 | 100 | QD | 100% TGI | <0.0001 |
| 1 | 75 | BID | 50% REG | <0.0001 |
| 2 | 10 | QD | 26% TGI | NS |
| 2 | 30 | QD | 35% TGI | NS |
| 2 | 50 | QD | 54% TGI | <0.0001 |
| 2 | 100 | QD | 79% TGI | <0.0001 |
| 2 | 50 | BID | 75% TGI | <0.0001 |
| 2 | 70 | BID, 5 days on, 2 days off | 88% TGI | <0.0001 |

TGI = tumor growth inhibition; REG = regression

TABLE 4B

| Vehicle | 30 mg/kg QD | 50 mg/kg QD | 100 mg/kg QD | 50 mg/kg BID |
|---|---|---|---|---|
| 2.4% +/− 0.2 | 3.7% +/− 0.4 | 4.2% +/− 0.3 | 5.9% +/− 0.5 | 6.8% +/− 0.4 |

### Results and Conclusions

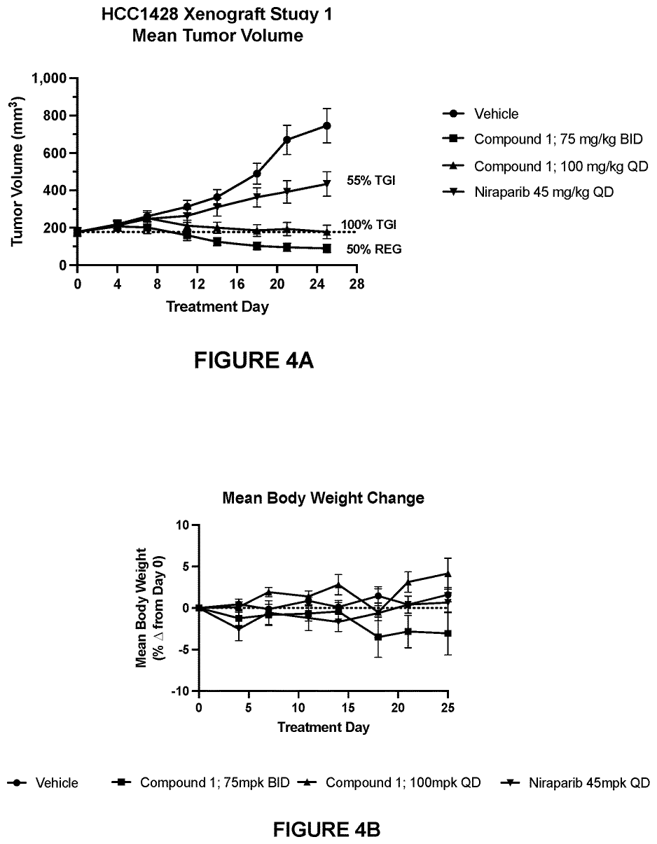

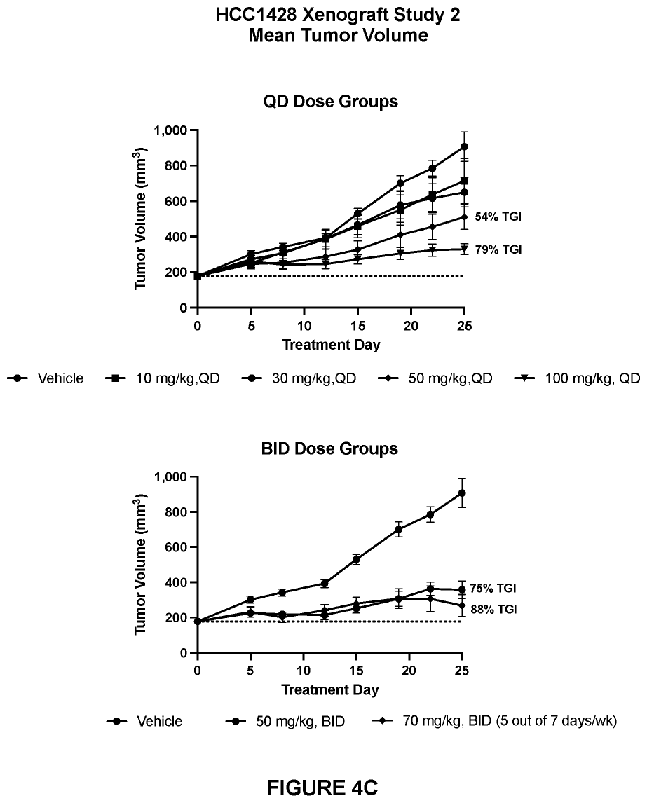

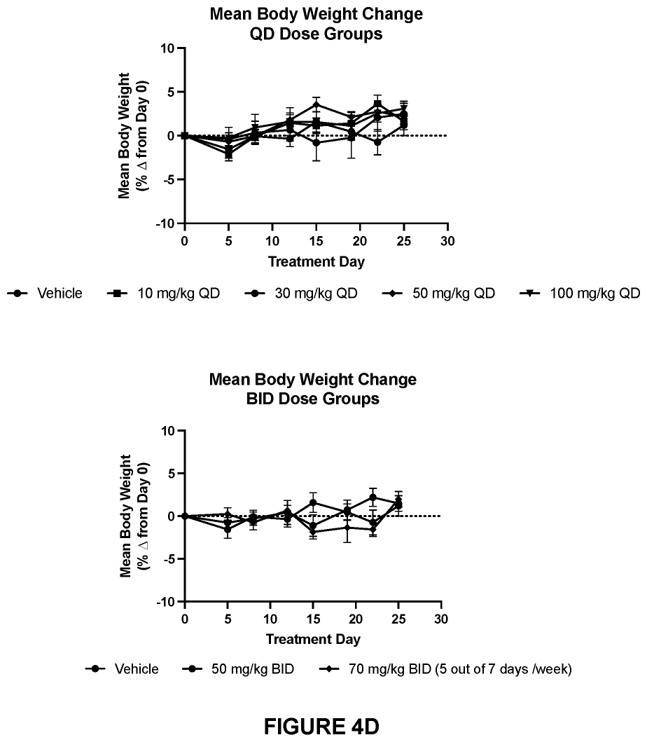

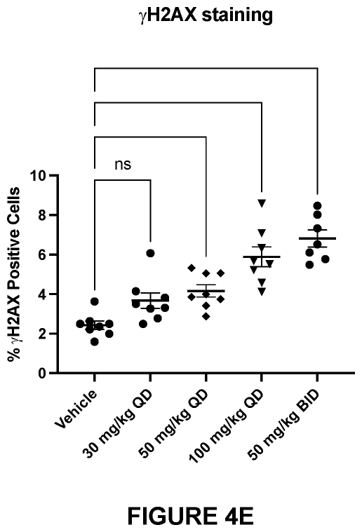

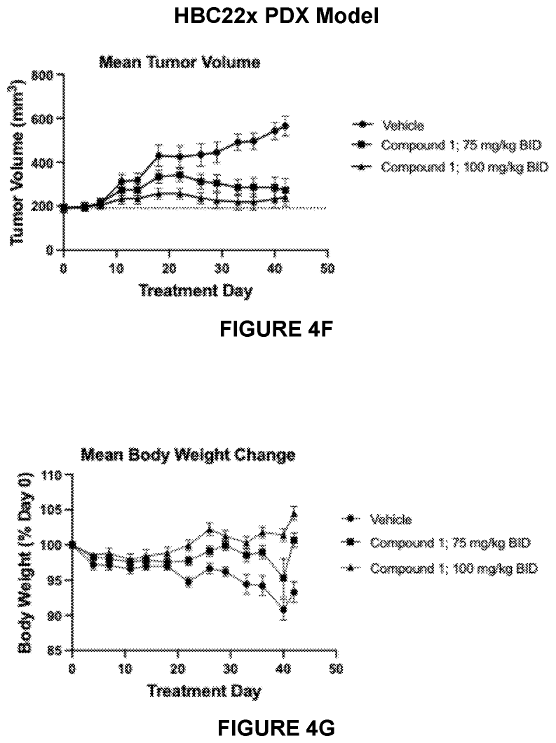

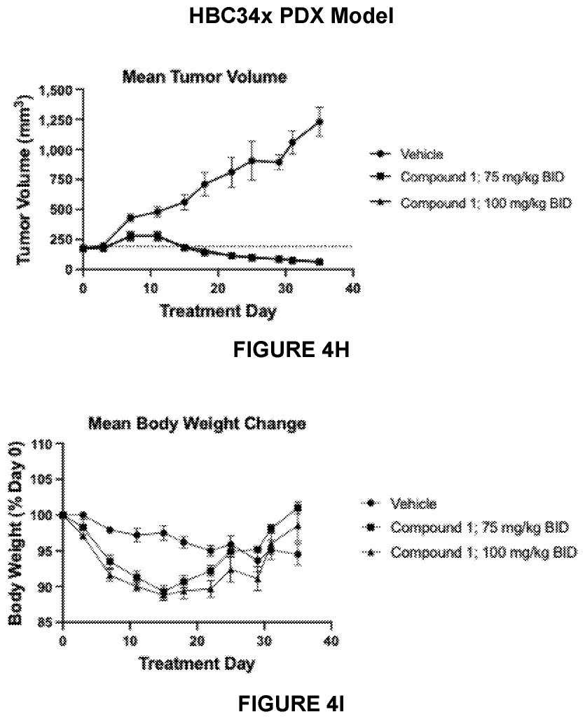

In study 1, significant tumor growth inhibition of the ER+HER2-BRCA2 mutant HCC1428 xenograft was observed with once or twice daily oral dosing of Compound 1. QD dosing of 100 mg/kg resulted in 100% mean TGI (p<0.0001), and BID dosing of 75 mg/kg resulted in 50% mean tumor regression (p<0.0001) (Table 4A, FIG. 4A). Animals dosed at 100 mg/kg QD showed a mean body weight increase over the treatment period. Animals dosed at 75 mg/kg BID showed a <4% mean body loss over the treatment period (FIG. 4B).

In study 2, once daily administration of Compound 1 showed dose-dependent TGI in the ER+HER2− BRCA2 mutant HCC1428 xenograft model, and doses of 50 and 100 mg/kg resulted in statistically significant 54% (p<0.0001) and 79% (p<0.0001) mean TGI, respectively (Table 4A, FIG. 4C). Robust TGI was also observed with BID dosing regimens, and 50 mg/kg BID and 70 mg/kg BID dosed the first 5 days of every 7-day cycle resulted in statistically significant 75% (p<0.0001) and 88% (p<0.0001) mean TGI, respectively (Table 4 A, FIG. 4C). Compound 1 was tolerated in all dose groups and limited, reversible effects on body weight were observed with no group showing >2% mean body weight loss during the treatment period (FIG. 4D). The significant TGI observed with 70 mg/kg BID dosing for the first 5 days of every 7-day cycle (88%, p<0.0001) demonstrated that efficacy can be maintained with a short dose holiday.

The expression of γH2AX was evaluated in formalin-fixed tumor tissue by immunohistochemistry. There was a statistically significant dose-dependent increase in the percentage of γH2AX positive cells in the tumor tissue, which correlated with efficacy (Table 4B, FIG. 4E). This observation suggests Compound 1 is inducing DNA double-strand breaks and/or replication catastrophe in cancer cells, which leads to anti-tumor efficacy.

### Example 4B: Efficacy of Compound 1 in Breast Cancer PDX Models

The anti-tumor activity and tolerability of Compound 1 was evaluated in two ER⁺ HER2⁻ breast cancer patient-derived xenograft (PDX) models, HBCx-22 and HBCx-34.

### Methods

The PDX models were established by subcutaneous inoculation of HBCx-22 or HBCx-34 cells into Hsd: Athymic Nude-Foxn1ₙᵤ mice. Breast tumor-bearing mice received estrogen diluted in drinking water (β-oestradiol, 8.5 mg/L) from the date of tumor inoculation until the end of the study to promote optimal growth of the ER⁺ cell line. Groups of 10 animals each were established by random assignment. Tumor volumes and body weights were measured twice weekly. Tumor volume and percent tumor growth inhibition (TGI) or percent tumor regression (REG) were calculated as described in Example 4A. Statistical analysis of end-of-study tumor volumes was performed using the Kruskal-Wallis test with Dunn's multiple comparisons test (HBCx-34) or Brown-Forsythe test with Dunnett's T3 multiple comparisons test (HBCx-22).

For the HBCx-22 study, Compound 1 treatment was initiated when the mean tumor volume of each group was approximately 190 mm³. Compound 1 was administered to mice orally twice daily (BID) for 42 days at a dose of 75 or 100 mg/kg based on body weight. In the HBCx-34 study, Compound 1 treatment was initiated when the mean tumor volume of each group was 176 mm³. Compound 1 was administered to mice orally BID for 35 days at doses of 75 or 100 mg/kg based on body weight.

### Results and Conclusions

In the HBCx-22 human breast cancer PDX model, Compound 1 showed robust tumor growth inhibition with 79% (p=0.0015) and 86% TGI (p<0.0001) when dosed at 75 mg/kg or 100 mg/kg BID, respectively (Table 4B, FIG. 4F). Treatment with Compound 1 was generally well tolerated as mean body weights of the drug treated groups were similar to or higher than the vehicle control group throughout the study, and animals dosed with 100 mg/kg BID showed higher mean body weights than animals dosed at 75 mg/kg BID (FIG. 4G).

In the HBCx-34 PDX model, Compound 1 showed robust 65% tumor regression when dosed at either 75 mg/kg or 100 mg/kg BID (p<0.001) (Table 4C, FIG. 4H). Compound 1 treatment resulted in a moderate and transient decrease in body weight which reached a maximum of approximately 11% at day 15. Body weight recovered for both Compound 1 treatment groups by end of study, with mean Day 35 body weights for both Compound 1 treatment groups greater than the vehicle group mean body weight (FIG. 4I).

TABLE 4C

| PDX | Compound 1 | Treatment | % TGI | |
|---|---|---|---|---|
| Model | Dose (mg/kg) | Regimen | or REG* | p-value |
| HBCx-22 | 75 | BID | 79% TGI | 0.0015 |
| HBCx-22 | 100 | BID | 86% TGI | <0.0001 |
| HBCx-34 | 75 | BID | 65% REG | <0.001 |
| HBCx-34 | 100 | BID | 65% REG | <0.001 |

TGI = tumor growth inhibition;
REG = regression

### Example 5: Efficacy of Compound 1 in Ovarian Cancer CDX and PDX Models

The anti-tumor activity and tolerability of Compound 1 was evaluated in two independent studies of a cell-line derived mouse xenograft model (CDX) using the RMUG-S human mucinous ovarian cancer cell line. Additionally, the antitumor efficacy of Compound 1 was evaluated in the T025OV high-grade serous ovarian cancer patient-derived xenograft (PDX) model.

### Methods

The CDX model was established by subcutaneous inoculation of 1×10⁷ viable RMUG-S cells into the right flank of NOD SCID or NOG mice. Groups of 8-10 animals were established by random assignment, and Compound 1 treatment was initiated when the mean tumor volume of each group was between 150-200 mm³. Compound 1 was administered to mice orally either once daily (QD) or twice daily (BID) for 32-50 days. Compound 1 was administered to mice continuously throughout the treatment period (7/7 days) or intermittently from Monday-Friday (5/7 days), or Monday-Thursday (4/7 days) during the treatment period.

T025OV tissue from a treatment naïve 53-year-old patient with Stage 1 serous ovarian cancer was obtained from Discovery Life Sciences (Huntsville, AL, Case ID #200000835) and was expanded by subcutaneous implantation into female NOG mice. For tumor development, mice were inoculated subcutaneously on the right flank with isolated T025OV cells at 1×10⁶ viable cells per mouse in 0.2 mL of DPBS mixed with Matrigel® Growth Factor Reduced Basement Membrane Matrix (1:1 ratio) (Corning®, Tewksbury, MA, USA, Cat #356237). Groups were established and drug treatment initiated when the mean tumor volume reached approximately 140-150 mm³. Compound 1 was administered to mice orally, twice daily (BID) for 50 days at dose level of 75 mg/kg based on body weight.

For all studies, tumor volumes and body weights were measured twice weekly. Tumor volume and percent tumor growth inhibition (TGI) or percent tumor regression (REG) were calculated as described in Example 4A. Statistical analysis of end-of-study tumor volumes for the RMUG-S studies was performed using the Brown-Forsythe and Welch ANOVA test with Dunnett's T3 multiple comparisons test. Statistical analysis of end-of-study mean tumor volumes for the T025OV model was performed using an unpaired t-test with Welch's correction.

TABLE 5

| | Com- | | TV | | |
|---|---|---|---|---|---|
| Ovarian | pound | | Measure- | | |
| Cancer | 1 Dose | Treatment | ment | % TGI | p- |
| Model | (mg/kg) | Regimen | Day | or REG | value |
| RMUG-S (1) | 25 | BID | 42 | 54% TGI | 0.01 |
| RMUG-S (1) | 50 | BID | 42 | 76% TGI | 0.0009 |
| RMUG-S (1) | 75 | BID | 42 | 93% TGI | 0.0003 |
| RMUG-S (2) | 100 | BID | 50 | 8% REG | <0.0001 |
| RMUG-S (2) | 200 | QD | 50 | 38% REG | <0.0001 |
| RMUG-S (2) | 100 | BID | 32 | 40% REG | <0.0001 |
| RMUG-S (2) | 100 | BID, 5 days | 32 | 16% REG | <0.0001 |
| | | on, 2 | | | |
| | | days off | | | |
| RMUG-S (2) | 100 | BID, 4 days | 32 | 18% REG | <0.0001 |
| | | on, 3 | | | |
| | | days off | | | |
| T025OV | 75 | BID | 50 | 77% TGI | 0.0002 |
| PDX | | | | | |

TGI = tumor growth inhibition;
REG = regression

### Results and Conclusions

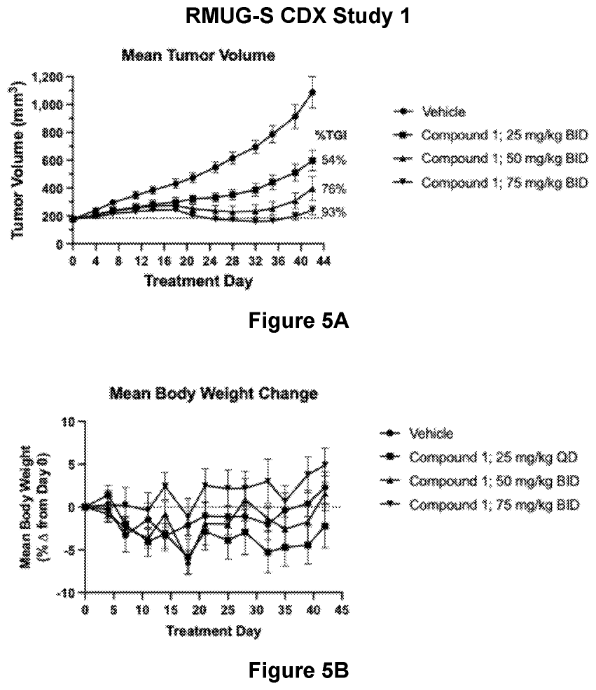

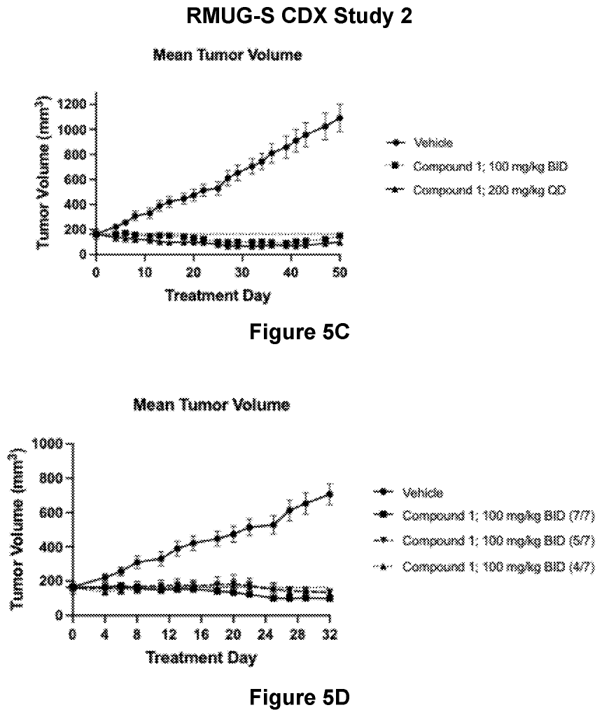

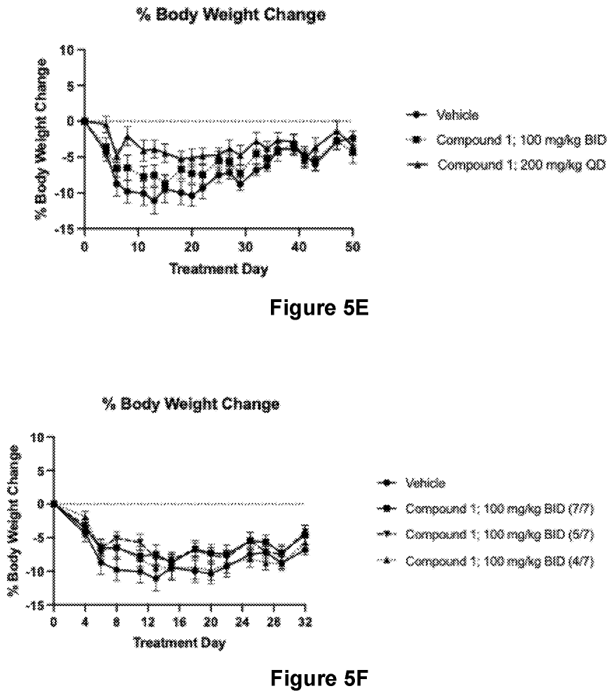

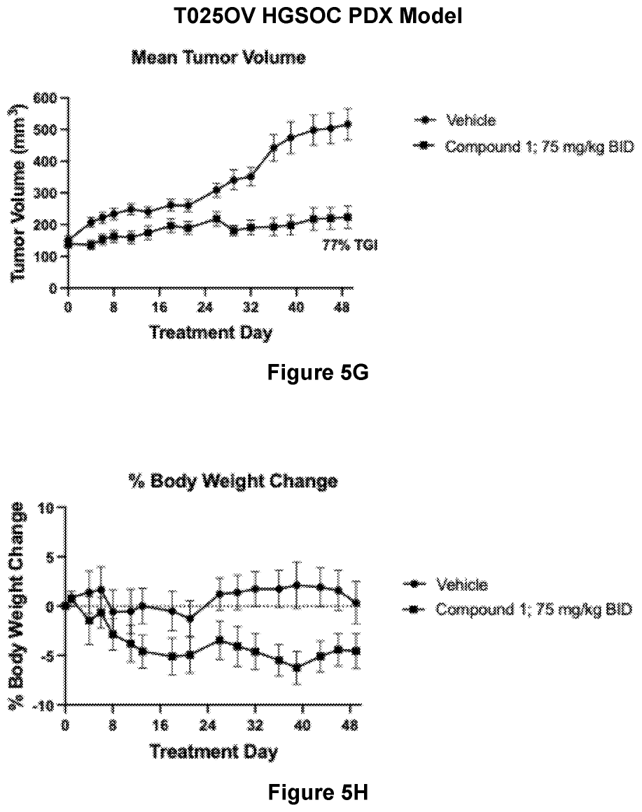

Oral treatment with Compound 1 resulted in statistically significant anti-tumor activity in the RMUG-S mucinous ovarian cancer CDX model. Dose-dependent inhibition of tumor growth was observed with twice daily dosing of Compound 1 with doses of 25, 50, or 75 mg/kg resulting in end-of-study mean TGI values of 54% (p<0.01), 76% (p<0.0009), and 93% (p<0.0003), respectively (Table 5, FIG. 5A). Compound 1 treatment was generally well tolerated, and mice in the highest dose group showed a mean 5% body weight gain over the treatment period (FIG. 5B). In a second study, robust TGI was observed with both BID dosing at 100 mg/kg and QD dosing at 200 mg/kg, with Day 50 end-of-study mean % REG values of 8% (p<0.0001) and 38% (p<0.0001), respectively (Table 5, FIG. 5C). Robust TGI was observed with continuous BID dosing (7/7), and intermittent dosing from Monday through Friday each week (5/7) or Monday through Thursday each week (4/7), with Day 32 end-of-study mean % REG values of 40% (p<0.0001), 16% (p<0.0001), and 18% (p<0.0001), respectively (Table 5, FIG. 5D). Compound 1 was generally well tolerated in the study with mean body weight loss that was less than or similar to the vehicle group throughout the treatment period (FIGS. 5E and 5F).

Compound 1 dosed at 75 mg/kg twice daily (BID) showed robust tumor growth inhibition in the T025OV human high-grade serous ovarian cancer (HGSOC) patient-derived xenograft model with a small and manageable mean decrease in body weight. Relative to the vehicle control, Compound 1 dosed at 75 mg/kg BID showed significant anti-tumor activity and resulted in a mean TGI of 77% (p<0.0002) (Table 5, FIG. 5G). Compound 1 treated group showed a mean 4.6% decrease in body weight over the same period (FIG. 5H).

### Example 6: Evaluation of Cellular Activity of Compound 1 in a PARP Inhibitor Resistant Cell Line

Compound 1 was evaluated for its ability to inhibit the proliferation of cancer cell lines that had developed resistance to a PARP inhibitor.

### Methods

The SNU601 gastric carcinoma cell line (Creative Bioarray, Cat #CSC-C9686L) was cultured in RPMI media containing glutamax, 10% fetal bovine serum, and 1% penicillin/streptomycin at 37° C., 5% CO₂. To generate a PARP inhibitor resistant cell line, an IC₂₀ concentration of niraparib was added to the culture media. The concentration of niraparib was slowly increased to the IC₉₀₋₁₀₀ in small increments over the course of 3-4 months until the cells adapted, developed resistance, and started proliferating in the presence of the high dose of niraparib.

Compound 1 and the PARP inhibitor niraparib were tested in cellular proliferation assays using the parental SNU601 cell line and the SNU601 cell line with acquired PARPi resistance. For the proliferation assays, cells were plated in 96-well plates (Corning costar 3603) at 2500-5000 cells/well in normal growth media. The next day, compound dissolved in vehicle (0.5% DMSO in media) was added to generate a 7- to 10-point concentration curve with half-log dilutions and starting concentrations of 10 or 31.6 μM. After 6 days of compound or vehicle treatment, the cells were fixed with a solution of 8% paraformaldehyde, 0.2% Triton X-100 for 20 minutes. After washing the cells with PBS, the nuclei were stained by the addition of DAPI (4′,6-diamidino-2-phenylindole) and incubation for 20 minutes. The number of nuclei in each well were counted by imaging the plates on an Image Xpress Pico automated cell imaging system (Molecular Devices). The percent proliferation after treatment was determined by normalizing the number of nuclei in treated wells to the vehicle-treated wells. Concentration-response-curves and IC₅₀ values for percent inhibition of proliferation were generated by non-linear regression analysis using GraphPad Prism 10 software.

TABLE 6

| | Compound 1 | Niraparib - PARPi |
|---|---|---|
| Cell Line | (μM) | (μM) |
| Parental SNU601 | 0.21 | 0.54 |
| Resistant SNU601 | 0.32 | 3.5 |

### Results and Conclusions

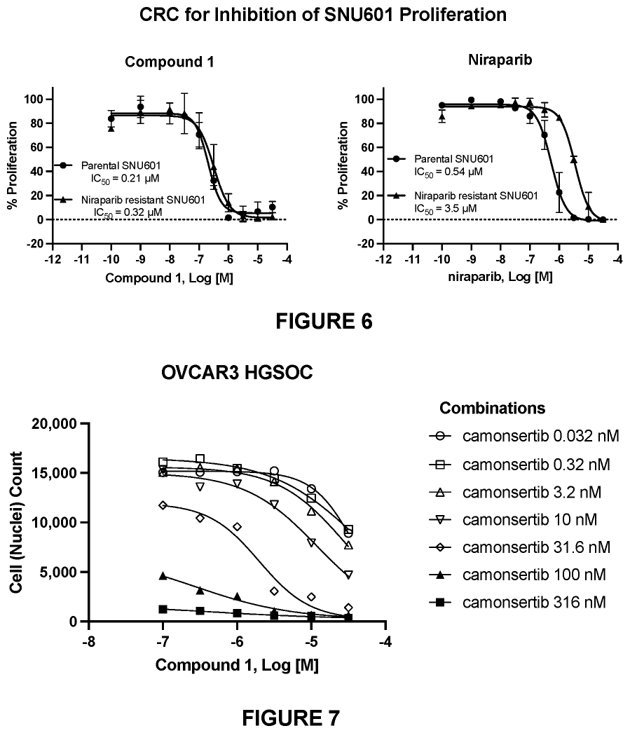

Compound 1 and the PARPi inhibitor niraparib showed similar potencies in inhibiting proliferation of the parental SNU601 gastric cancer cell line. The PARPi inhibitor niraparib showed a 7× loss in potency to the PARPi resistant SNU601 cell line versus the parental line. Conversely, Compound 1 maintained near complete sensitivity to the PARPi resistant SNU601 cell line and showed 10× better potency than niraparib (Table 6, FIG. 6).

These data demonstrate that a cancer cell that develops resistance to a PARP inhibitor can still be highly sensitive to Compound 1. Similarly, the HCC1428 breast cancer cell line was derived from a patient that had developed resistance to platinum-based therapies, and it showed good sensitivity to Compound 1 (Table 3). Compound 1 can be a beneficial cancer therapy for patients that have developed resistance to other cancer therapies such as PARP inhibitors or platinum-based therapies.

### Example 7: Evaluation of Cellular Activity of Compound 1 in Combination with an ATR Inhibitor in the OVCAR3 Ovarian Cancer Cell Line

The cellular potency and activity of Compound 1 was evaluated in combination with camonsertib (RP-3500), an inhibitor of the ataxia telangiectasia and Rad3-related (ATR) protein.

### Methods

The combinatorial activity of Compound 1 plus camonsertib was evaluated in the OVCAR3 human high-grade serous ovarian cancer (HGSOC) cell line in a 6-day growth assay. OVCAR3 cells were obtained from American Type Culture Collection (ATCC) and maintained in RPMI+20% FBS+1% penicillin/streptomycin+0.01 mg/mL bovine insulin in a humidified incubator at 37° C., 5% CO₂. OVCAR3 cells were seeded at 5,000 cells/well in 100 μL of growth medium in a 96-well plate (Corning costar 3603) and allowed to adhere overnight at 37° C. with 5% CO₂. The following day, serial dilutions of Compound 1 and camonsertib were prepared and added to the corresponding wells of a 96-well plate. A solution of 0.1% DMSO in medium was used as the negative control. Cells were incubated with compound for 6 days at 37° C. with 5% CO₂, then fixed, stained, and counted as described in Example 3. The number of nuclei in each well were counted by imaging the plates on an ImageXpress Pico automated cell imaging system (Molecular Devices). Concentration-response-curves and IC₅₀ values were generated by non-linear regression analysis using a 4-parameter variable slope fit in GraphPad Prism 10 software. Potential synergy between Compound 1 and camonsertib was assessed using Zero interaction potency (ZIP), Highest Single Agent (HSA), Bliss, and Loewe synergy models to find the most synergistic area score, where scores >10 indicate that the interaction between the two drugs is likely to be synergistic (Ianevski et al. 2020).

TABLE 7A

| | Camonsertib (RP-3500) | Compound 1 IC₅₀ (μM) |
|---|---|---|
| Cell Line | Concentration (nM) | 6-Day Nuclei Count |
| OVCAR3 | 0.032 | 39 |
| | 0.32 | 44 |
| | 3.2 | 29 |
| | 10 | 12 |
| | 32 | 2 |
| | 100 | 0.27 |

TABLE 7B

| Synergy | | Synergy |
|---|---|---|
| Model | Metric | Score |
| ZIP | camonsertib (10-31.6 nM), Compound 1 (3.2-10 μM) | 36.7 |
| HSA | camonsertib (10-31.6 nM), Compound 1 (10-31.6 μM) | 42.2 |
| Loewe | camonsertib (3.16-10 nM), Compound 1 (10-31.6 μM) | 34.6 |
| Bliss | camonsertib (10-31.6 nM), Compound 1 (3.2-10 μM) | 37.5 |

### Results and Conclusions

Compound 1 in the presence of the lowest tested concentration of camonsertib (0.032 nM) resulted in a growth inhibitory IC₅₀ value of 39 μM. When combined with increasing concentrations of camonsertib, Compound 1 potency was enhanced, shifting the IC 50 values to 44, 29, 12, 2, and 0.27 μM for camonsertib concentrations of 0.32, 3.2, 10, 32, and 100 nM, respectively (Table 7A, FIG. 7). This cellular study demonstrated a combination benefit when an ATR inhibitor, camonsertib, was added to Compound 1 treatment in a human high-grade serous ovarian cancer cell line.

The results from the 6-day growth assay were analyzed using multiple synergy models. Zip, HSA, Bliss, and Loewe models calculated most synergistic area scores corresponding to 36.7, 42.2, 34.6, and 37.5, respectively and indicated overall broad synergy at different concentrations (Table 7B). Taken together, this cellular study and subsequent synergy analyses demonstrated a synergistic combination benefit between Compound 1 and camonsertib in the OVCAR3 human high-grade serous ovarian cancer cell line. These results indicate that combination of Compound 1 with other therapeutic agents can be beneficial for the treatment of cancer.

### Example 8: Evaluation of Cellular Activity of Compound 1 in Combination with a Topoisomerase I Inhibitor

The cellular potency and activity of Compound 1 in combination with the topoisomerase I inhibitor, exatecan, was evaluated in the human breast cancer cell lines HCC1954 and HCC1806 and the colorectal cancer cell line GP2D.

### Methods

The activity of Compound 1 in combination with exatecan was evaluated in the HCC1954, HCC1806, and GP2D cancer cell lines in a 6-day growth assay. HCC1954 (Cat #CRL-2338) and HCC1806 (Cat #CRL-2335) cells were obtained from American Type Culture Collection (ATCC) and maintained in ATCC modified RPMI (Cat #30-2001)+10% FBS (Cat #A3160502)+1% penicillin/streptomycin (Cat #15140-122) in a humidified incubator at 37° C., 5% CO₂. GP2D cells were obtained from ECACC (Cat #95090714-1VL) and maintained in DMEM+GlutaMAX (Cat #10566-016)+10% FBS (Cat #A3160502)+1% penicillin/streptomycin (Cat #15140-122). Cells were seeded at 2,500 cells/well (HCC1954 and GP2D) or 2,000 cells/well (HCC1806) in 100 μL of growth medium in a 96-well plate (Corning costar Cat #3603) and allowed to adhere overnight at 37° C. with 5% CO₂. The next day, serial dilutions of Compound 1 and exatecan were prepared and added to the corresponding wells of a 96-well plate. A solution of 0.1% DMSO in medium was used as the negative control. Cells were incubated with compound for 6 days at 37° C. with 5% CO₂, then fixed, stained, and counted as described in Example 3. Concentration response-curves and IC₅₀ values were generated by non-linear regression analysis using a 4-parameter variable slope fit in GraphPad Prism 10 software. Potential synergy between Compound 1 and exatecan was assessed using Zero interaction potency (ZIP), Highest Single Agent (HSA), Bliss, and Loewe synergy models to find the most synergistic area score, where scores >10 indicate that the interaction between the two drugs is likely to be synergistic (Ianevski et al. 2020).

### Results and Conclusions

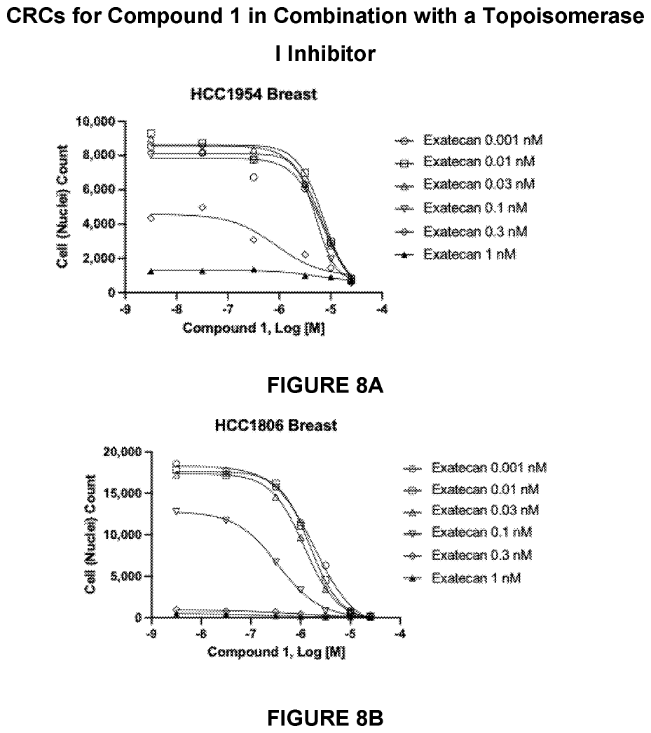

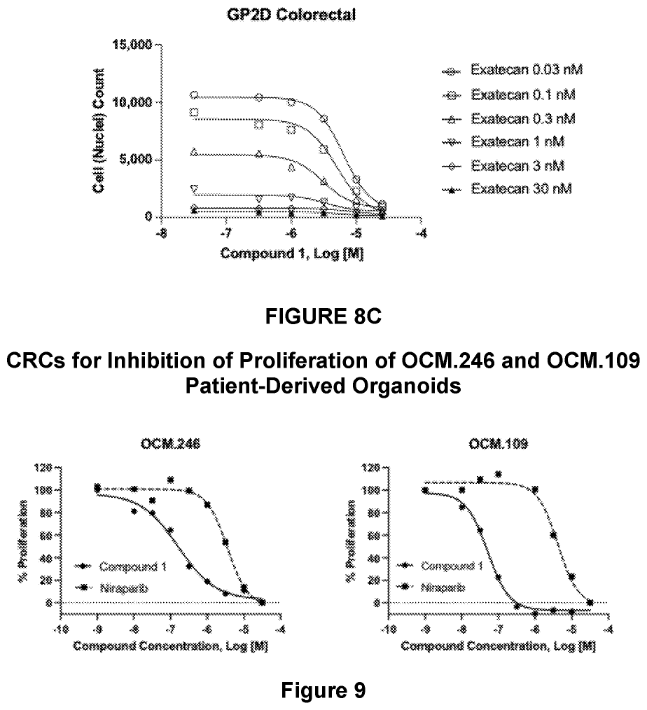

In HCC1954 cells, Compound 1 in the presence of the lowest tested concentration of exatecan (0.001 nM) showed a growth inhibitory IC₅₀ value of 6.9 μM. Increasing concentrations of exatecan in combination with Compound 1 improved the potency, shifting the Compound 1 IC₅₀ value to 0.9 μM in the presence of 0.3 nM exatecan (Table 8A, FIG. 8A). In the breast cancer cell line, HCC1806, the Compound 1 growth inhibition IC 50 value at the lowest exatecan concentration tested was 1.9 μM. In combination with 0.1 nM exatecan, the potency of Compound 1 was improved to 0.3 μM (Table 8C, FIG. 8B). In the GP2D colorectal cell line, Compound 1 displayed an IC₅₀ value of 6 μM at the lowest exatecan concentration tested of 0.03 nM. In combination with 1 nM exatecan, the potency of Compound 1 was improved resulting in an IC₅₀ value of 3.1 μM (Table 8E, FIG. 8C).

This in vitro cellular combination data demonstrated a benefit when the topoisomerase I inhibitor, exatecan, was added to Compound 1 treatment in human breast and colorectal cancer cell lines. The results from the 6-day growth assay were analyzed using multiple synergy models including Zip, HSA, Bliss, and Loewe. The models calculated the most synergistic area scores and indicated overall broad synergy with scores for Zip, HSA, Bliss and Loewe shown respectively for HCC1954 (18.2, 27, 17.3, 19.5), HCC1806 (17.8, 25.6, 10.1, 17.6) and GP2D (12.3, 18.4, 8.1, 12.7) as described in Tables 8B, 8D, and 8F, respectively. Altogether, this cellular study and subsequent synergy analyses demonstrated a synergistic combination benefit between Compound 1 and exatecan in the HCC1954, HCC1806, and GP2D cancer cell lines. These results demonstrate that the combination of Compound 1 with a topoisomerase I inhibitor can be beneficial for the treatment of cancer.

TABLE 8A

| | Exatecan | Compound 1 |
|---|---|---|
| Cell Line | Concentration (nM) | IC₅₀ (μM) |
| HCC1954 | 0.001 | 6.9 |
| | 0.01 | 7.2 |
| | 0.03 | 6.6 |
| | 0.1 | 5.2 |
| | 0.3 | 0.9 |
| | 1 | Not able to calculate |

TABLE 8B

| Synergy | | |
|---|---|---|
| Model | Metric | Score |
| ZIP | Exatecan (0.1-0.3 nM), Compound 1 (1-3 μM) | 18.2 |
| HSA | Exatecan (0.1-0.3 nM), Compound 1 (1-3 μM) | 27 |
| Loewe | Exatecan (0.1-0.3 nM), Compound 1 (0.3-1 μM) | 17.3 |
| Bliss | Exatecan (0.1-0.3 nM), Compound 1 (1-3 μM) | 19.5 |

TABLE 8C

| Cell | Exatecan | Compound 1 |
|---|---|---|
| Line | Concentration (nM) | IC₅₀ (μM) |
| HCC1806 | 0.001 | 1.9 |
| | 0.01 | 1.5 |
| | 0.03 | 1.2 |
| | 0.1 | 0.3 |
| | 0.3 | Not able to calculate |

TABLE 8D

| Synergy | | |
|---|---|---|
| Model | Metric | Score |
| ZIP | Exatecan (0.03-0.1 nM), Compound 1 (0.3-1 μM) | 17.8 |
| HSA | Exatecan (0.03-0.1 nM), Compound 1 (1-3 μM) | 25.6 |
| Loewe | Exatecan (0.03-0.1 nM), Compound 1 (1-3 μM) | 10.1 |
| Bliss | Exatecan (0.03-0.1 nM), Compound 1 (1-3 μM) | 17.6 |

TABLE 8E

| Cell | Exatecan | Compound 1 |
|---|---|---|
| Line | Concentration (nM) | IC₅₀ (μM) |
| GP2D | 0.03 | 6.3 |
| | 0.1 | 5.1 |
| | 0.3 | 3.1 |
| | 1 | 3.3 |
| | 3 | 8.1 |
| | 30 | 5.4 |

TABLE 8F

| Synergy | | |
|---|---|---|
| Model | Metric | Score |
| ZIP | Exatecan (0.1-0.3 nM), Compound 1 (1-3 μM) | 12.3 |
| HSA | Exatecan (0.1-0.3 nM), Compound 1 (1-3 μM) | 18.4 |
| Loewe | Exatecan (0.1-0.3 nM), Compound 1 (1-3 μM) | 8.1 |
| Bliss | Exatecan (0.1-0.3 nM), Compound 1 (1-3 μM) | 12.7 |

### Example 9: Inhibition of Cellular Proliferation by Compound 1 Using Patient-Derived Ovarian Cancer Models (OCM)

Compound 1 was evaluated for its ability to inhibit proliferation of two PARP inhibitor-resistant, patient-derived high-grade serous ovarian cancer models (OCMs), OCM.109 and OCM.246. OCM.246 was derived from a patient with a germline BRCA2 mutation who received Olaparib maintenance monotherapy prior to biopsy sampling. Molecular analysis of OCM.246 cells showed an intragenic BRCA2 reversion mutation predicted to restore the open reading frame of BRCA2. Cellular analysis using RAD51 foci formation demonstrated that OCM.246 is homologous recombination proficient (HRP), whereas OCM.109 is homologous recombination deficient (HRD) (Coulson-Gilmer, 2021, J Exp Clin Cancer Res).

### Methods

Ex vivo ovarian cancer models (OCMs) were established from the ascites of high-grade serous ovarian cancer patients (Coulson-Gilmer, 2021, J Exp Clin Cancer Res) and grown in vitro as 2D cell lines. OCMs were cultured in an incubator at 37° C. in 5% CO₂ in OCMI media. In brief, OCMI media is a 50:50 mix of Nutrient Mixture Ham's F-12 (Millipore Sigma, Burlington, MA, USA, Cat #N6760) and Medium 199 (Life Technologies, Thermo Fisher Scientific, Waltham, MA, USA, Cat #11825015) supplemented with 5% FBS (Life Technologies, Thermo Fisher Scientific, Waltham, MA, USA, Cat #A5670402) for OCM.109 or 5% HyClone FBS (Cytiva, Marlborough, MA, USA, Cat #SH30071.03) for OCM.246, 2 mM glutamine (Cat #G7513-100ML), 100 U/mL penicillin-streptomycin (Cat #P0781-100ML), 10 mM HEPES at pH 7.4 (Cat #H4034), 20 μg/mL human insulin (Cat #19278), 0.01 μg/mL hEGF (Cat #E9644), 0.5 μg/mL hydrocortisone (Cat #H0888), 10 μg/mL transferrin (Cat #T8158), 1 μg/mL 3,3′,5-triiodo-2-thyronine (Cat #T2877), 5 μg/mL o-phosphorylethanolamine (Cat #P0503), 8 ng/ml selenous acid (Cat #211176), 0.5 ng/ml 17β-estradiol (Cat #e2758), linoleic acid (Cat #L1012), 1.75 μg/mL hypoxanthine (Cat #H9636), 0.05 μg/mL α-lipoic acid (Cat #T1395), 0.05 μg/mL cholesterol (Cat #C3045), 0.003 μg/mL α-tocopherol phosphate disodium salt (Cat #T2020), 0.025 μg/mL ergocalciferol (Cat #E5750), 3.5 μg/mL choline chloride (Cat #C7527), 0.33 μg/mL folic acid (Cat #F8758), 1.166 μg/mL vitamin B12 (Cat #V6629), 0.08 μg/mL thiamine HCl (Cat #T1270), 4.5 μg/mL myo-inositol (Cat #17508), 0.125 μg/mL D-(−)-ribose (Cat #R9629), 0.0125 μg/mL para-aminobenzoic acid (Cat #A9878), 1.25 mg/mL BSA (Cat #A2153), 0.085 μg/mL xanthine (Cat #X4002), 25 ng/ml cholera toxin (Cat #C8052), L-glutathione reduced (Cat #G6013) (all from Millipore Sigma, Burlington, MA, USA), and 0.0246 μg/mL all-trans retinoic acid (Axon Medchem, Reston, VA, USA, Cat #R3321), 0.012 μg/mL L-ascorbic acid (TCI Chemicals, Portland, OR, USA, Cat #TCI-A0537-25G), and 0.075 μg/mL uracil (TCI Chemicals, Portland, OR, USA, Cat #TCI-U0031-25G).

Compound 1 IC₅₀ values for inhibition of cell proliferation were determined by imaging and counting the number of nuclei after compound treatment. Cells were harvested with trypsin (Thermo Fisher Scientific, Waltham, MA, USA, Cat #25200-056) and plated in Sarstedt 96-well Cell+ Tissue Culture Plates (Thermo Fisher Scientific, Waltham, MA, USA, Cat #NC9624222) at 5,000 cells/well (OCM.109) or 10,000 cells/well (OCM.246) in 100 μL of OCMI medium and incubated overnight at 37° C., 5% CO₂. The following day, 1000× Compound 1 and niraparib concentration curves were prepared by serial dilution in 100% DMSO. The 1000× concentration was diluted 1:200 into growth media to generate a 5× concentration curve in 0.5% DMSO/growth medium before addition of 25 μL of the 5× compound to the wells. Control wells received 25 μL of 0.5% DMSO in media or 10 μM of Compound 1. The plates were mixed and incubated for 6 days at 37° C., 5% CO2 before fixing and staining the cells for nuclei counting as described in Example 3.

Four fields in each well were acquired using the DAPI channel at 10× magnification. Using high-content software (Cell Reporter Xpress), the nuclei were detected and counted. The percent proliferation after treatment was determined by normalizing the number of nuclei in the treated wells to the DMSO and 10 μM Compound 1 control wells. Concentration response curves and IC₅₀ values for percent inhibition of proliferation were generated by non-linear regression analysis using a least squares fit of the log (inhibitor) vs. response-Variable slope (four parameters) curve using GraphPad Prism 10 software (GraphPad Software, Boston, MA, USA).

TABLE 9

| Ovarian Cancer | Compound 1 | Niraparib |
|---|---|---|
| Model | IC₅₀ (μM) | IC₅₀ (μM) |
| OCM.246 | 0.162 | 3.56 |
| OCM.109 | 0.051 | 4.17 |

### Results and Conclusions

The potency of Compound 1 for inhibiting cell proliferation in two PARP inhibitor-resistant patient-derived ovarian cancer models, OCM.246 and OCM.109, was compared to the potency of the PARP inhibitor, niraparib, by measuring nuclei count after six days of treatment. The concentration response curves for percent proliferation are shown in FIG. 9, and the IC 50 values for inhibition of proliferation are shown in Table 9. Compound 1 displayed potent and concentration-dependent inhibition of proliferation, with IC₅₀ values of 0.162 and 0.051 μM for OCM.246 and OCM.109, respectively. Compound 1 showed high potency that was >20-fold better than niraparib against the OCM.246 HRP cells that contained a BRCA2 reversion mutation and also showed high potency that was >80-fold better than niraparib against the OCM.109 HRD cells.

### Example 10: Efficacy of Compound 1 in Combination with Abemaciclib and Fulvestrant in the ER⁺ HER2⁻ HCC1428 Breast Cancer CDX

The anti-tumor activity and tolerability of Compound 1 alone or in combination with the CDK4/6 inhibitor, abemaciclib, and/or the endocrine therapy, fulvestrant, was evaluated in a cell-line derived mouse xenograft model (CDX) using the ER⁺ HER2-HCC1428 human breast cancer cell line.

### Methods

The CDX model was established by subcutaneous inoculation of HCC1428 cells into the right flank of BALB/c nude mice. Mice were injected subcutaneously with estradiol benzoate one day prior to cell inoculation and then twice per week thereafter to promote optimal growth of the ER⁺ cell line. Groups of 9 animals each were established by random assignment. Tumor volumes and body weights were measured twice weekly. Tumor volume and percent tumor growth inhibition (TGI) or percent tumor regression (REG) were calculated as described in Example 4A. End-of-study Day 55% TGI or REG was calculated relative to the Day 25 mean TV from the vehicle group. Statistical analysis of the Day 25 tumor volumes was performed using the Brown-Forsythe and Welch ANOVA test.

Compound treatment was initiated when the mean tumor volume of each group was 184 mm³. Compound 1 was administered to mice orally for 25 days once daily (QD) at a dose of 100 mg/kg based on body weight. Abemaciclib was administered orally at a dose of 30 mg/kg QD for 25 days based on body weight and fulvestrant was administered subcutaneously at a dose of 5 mg/mouse once per week (QW). Following the dosing period of 25 days, an observation period was conducted for Group 5 (Compound 1+abemaciclib), Group 7 (abemaciclib+fulvestrant), and Group 8 (Compound 1+abemaciclib+fulvestrant) with continued twice weekly measurement of tumor volume and body weight measurements but no additional administration of compound.

### Results and Conclusions

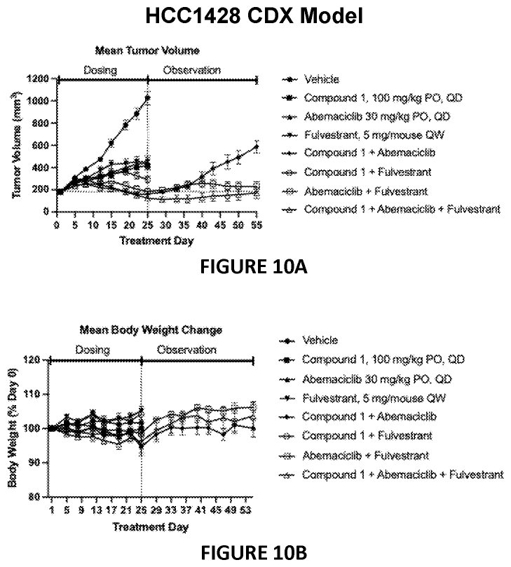

There was significant monotherapy anti-tumor activity in the HCC1428 xenograft with mean % TGI values of 72% (p<0.0001), 70% (p<0.0001), and 68% (p<0.0001) for Compound 1, abemaciclib, and fulvestrant, respectively (p<0.0001) (Table 10A, FIG. 10A).

Compound 1 in combination with abemaciclib showed increased tumor growth inhibition compared to monotherapy, resulting in statistically significant anti-tumor activity on Day 25 with mean tumor regression of 13% (p<0.0001) (Table 10A, FIG. 10A). When administered in combination, Compound 1+fulvestrant showed a tumor growth inhibition of 87% TGI (p<0.0001) which was better than each monotherapy alone. The triple combination of Compound 1+abemaciclib+fulvestrant exhibited the maximum anti-tumor activity during the treatment period with a mean tumor regression of 32% (p<0.0001) (Table 10A, FIG. 10A). To monitor the durability of tumor growth inhibition, a 30-day observation period was conducted without dosing for Groups 5, 7 and 8. Animals in the Compound 1+abemaciclib treated group (Group 5) showed moderate tumor re-growth with a final TGI of 52% at the Day 55 end of the study (Table 10B and FIG. 10A). Minimal tumor growth was observed for the abemaciclib+fulvestrant treated group (Group 7) which displayed 100% TGI on Day 55. The triple combination of Compound 1+abemaciclib+fulvestrant (Group 8) exhibited the most durable tumor growth inhibition at the end of the observation period with 7% regression (Table 10B and FIG. 10A).

Compound 1 was well tolerated in all dose groups as a monotherapy or in combinations. Body weight reductions were minimal with none of the groups showing >6% mean body weight loss during the treatment period (FIG. 10B).

These results highlight the robust anti-tumor efficacy of Compound 1 in combination with the CDK4/6 inhibitor abemaciclib and the endocrine therapy fulvestrant in the ER⁺ HER2⁻ HCC1428 breast cancer CDX model.

TABLE 10A

| | Compound | Abemaciclib | Fulvestrant | Day 25 | |
|---|---|---|---|---|---|
| | 1 Dose | Dose | Dose | % TGI | |
| Group | (mg/kg) | (mg/kg) | (mg/mouse) | or REG | p-value |
| 1-Vehicle | — | — | — | — | — |
| 2-Compound 1 | 100 | — | — | 72% TGI | <0.0001 |
| 3-Abemaciclib | — | 30 | — | 70% TGI | <0.0001 |
| 4-Fulvestrant | — | — | 5 | 68% TGI | <0.0001 |
| 5-Compound 1 + | 100 | 30 | — | 13% REG | <0.0001 |
| Abemaciclib | | | | | |
| 6-Compound 1 + | 100 | — | 5 | 87% TGI | <0.0001 |
| Fulvestrant | | | | | |
| 7-Abemaciclib + | — | 30 | 5 | 100% TGI | <0.0001 |
| Fulvestrant | | | | | |
| 8-Compound 1 + | 100 | 30 | 5 | 32% REG | <0.0001 |
| Abemaciclib + | | | | | |
| Fulvestrant | | | | | |

TGI = tumor growth inhibition;
REG = regression

TABLE 10B

| | Compound 1 | Abemaciclib | Fulvestrant | Day 55 |
|---|---|---|---|---|
| | Dose | Dose | Dose | % TGI |
| Group | (mg/kg) | (mg/kg) | (mg/mouse) | or REG |
| 5-Compound 1 + | 100 | 30 | — | 52% TGI |
| Abemaciclib | | | | |
| 7-Abemaciclib + | — | 30 | 5 | 95% TGI |
| Fulvestrant | | | | |
| 8-Compound 1 + | 100 | 30 | 5 | 7% REG |
| Abemaciclib + | | | | |
| Fulvestrant | | | | |

TGI = tumor growth inhibition;
REG = regression

### Example 11: Efficacy of Compound 1 in Combination with the ATR Inhibitor Camonsertib (RP-3500) in the Triple Negative Breast Cancer Cell Line HCC1806

The anti-tumor activity and tolerability of Compound 1 in combination with the ATR inhibitor, camonsertib (RP-3500), was evaluated in a cell-line derived mouse xenograft (CDX) model using the BRCA wild-type triple-negative breast cancer cell line HCC1806.

### Methods

HCC1806 cells were cultured in RPMI medium containing 10% FBS (Thermo Fisher Scientific Cat #A31605-02) and penicillin+streptomycin (Thermo Fisher Scientific Cat #15070-063). The CDX model was established by subcutaneous inoculation of 1×10⁶ HCC1806 cells/mouse into the right flank of Athymic nude mice (The Jackson Laboratory, Bar Harbor, ME, USA, Cat #002019). Groups of 9 animals each were established by random assignment, and Compound 1 treatment was initiated when the mean tumor volume was approximately 150 mm³. Compound 1 was administered to mice at 200 mg/kg to mice orally once daily from Monday through Friday throughout the treatment period and camonsertib (RP-3500) was administered at 15 mg/kg orally once daily from Monday through Wednesday throughout the treatment period (Table 11).

Tumor volumes and body weights were measured twice weekly. Tumor volume and percent tumor growth inhibition (TGI) or percent tumor regression (REG) were calculated as described in Example 4A. Statistical analysis of end-of-study tumor volumes relative to the vehicle group tumor volume was performed using Kruskal-Wallis Anova test with Dunn's multiple comparisons test.

TABLE 11

| | Compound 1 | RP-3500 Dose | % TGI | |
|---|---|---|---|---|
| Group | Dose (mg/kg) | (mg/kg) | or REG | p-value |
| 1-Vehicle | — | — | — | — |
| 2-Compound 1 | 200 | — | 0% TGI | n.s. |
| 3-RP-3500 | — | 15 | 41% TGI | n.s. |
| 4-Compound 1 + | 200 | 15 | 76% REG | 0.001 |
| RP-3500 | | | | |

n.s. = not significant;
TGI = tumor growth inhibition;
REG = regression

### Results and Conclusions

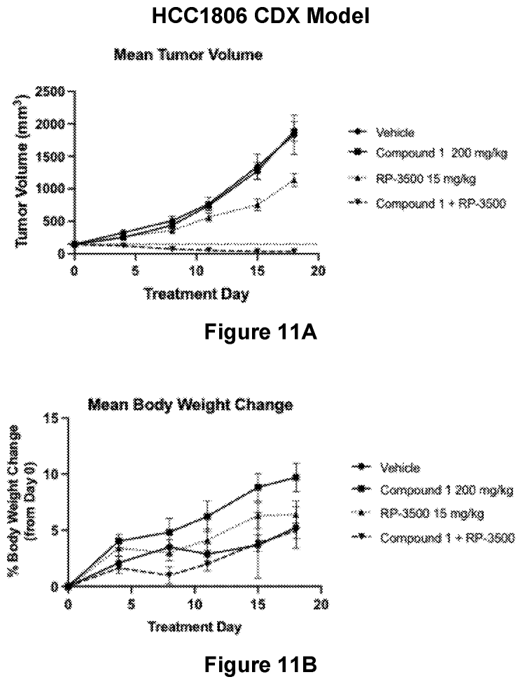

Oral treatment with Compound 1 as a monotherapy resulted in no tumor growth inhibition in the HCC1806 CDX model (Table 11, FIG. 11A). Oral treatment with the ATR inhibitor RP-3500 resulted in a non-significant 41% mean TGI. Compound 1 showed a combination effect with RP-3500, resulting in statistically significant anti-tumor activity with a mean tumor regression of 76% (Table 11, FIG. 11A). These results highlight the robust anti-tumor efficacy of Compound 1 in combination with the ATR inhibitor RP-3500 in a BRCA wild-type triple-negative breast cancer model. Compound 1 was generally well tolerated as a monotherapy and in combination with RP-3500 with both treatment groups showing body weight gains during the treatment period (FIG. 11B).

### Example 12: a Phase ½ Study of PARG Inhibitor Compound 1 in Patients with Advanced Solid Malignancies

Study Title: A Phase ½ Study of PARG inhibitor Compound 1 in Patients with Advanced Solid Malignancies

Indication: Patients with advanced solid malignancies that have progressed following standard therapy

Background and Study Rationale: A hallmark of many cancer cells is replication stress, which is characterized by the slowing or stalling of replication forks during the DNA replication process, leading to the accumulation of damaged DNA. The cellular response to replication stress is the activation of cell-cycle checkpoints and the DNA damage response (DDR) pathway to arrest the cell cycle and promote repair of the damaged DNA. Poly (ADP) ribose glycohydrolase (PARG) plays a critical role in DDR with genetic depletion or inhibition by reference compounds resulting in increased numbers of single-strand breaks (SSBs) and double-strand breaks (DSBs) and reduced kinetics of break repair. In addition, under conditions of replication stress in cancer cells, PARG depletion or inhibition has been shown to inhibit proliferation and arrest cells in the S or G2 phase of the cell cycle and/or induce apoptosis alone or in combination with DNA damaging agents or replication stress inducers. The replication stress response represents a cancer-specific vulnerability, which can be targeted by PARG small molecule inhibition.

Study Design Overview: This is a first in human (FIH), two-part, open-label, multicenter, dose escalation and dose expansion study designed to evaluate the safety, tolerability, pharmacokinetics (PK), pharmacodynamics (PDx), and anti-tumor activity of Compound 1, a novel reversible small molecule inhibitor of PARG. In addition, this study will determine the recommended dose(s) for expansion (RDE) and/or recommended Phase 2 dose (RP2D) to further assess the objective response to Compound 1 therapy in patients with advanced solid tumors meeting the expansion cohort eligibility criteria.

Phase 1 of this protocol is an open-label, dose-escalation, and cohort expansion study in patients with advanced recurrent or metastatic cancer that has progressed after prior standard therapies. Phase 1 will be conducted in two stages: In Part 1A (Dose-Escalation): Patients will be enrolled in dose cohorts using a Bayesian optimal interval (BOIN) design. In Part 1B, additional patients will be enrolled in Dose Expansion cohorts to further evaluate safety, PK, PDx, preliminary anti-tumor activities or, in specific sub-populations of patients, to optimize RP2D.

All patients will be required to provide an archived or fresh tumor biopsy or liquid biopsy blood sample for retrospective central review and exploratory analyses of potential biomarkers of activity and response.

Safety will be monitored through the collection of adverse events (AEs), physical examinations, vital signs, assessment of clinical laboratory values, and electrocardiograms (ECGs). Safety data will be reviewed throughout the study by the study Medical Monitor and by the study Safety Review Committee (SRC), comprised of the Sponsor's clinical study team and study investigators from sites participating in this study.

Pharmacokinetics will be assessed by evaluating plasma levels of Compound 1 in Cycle 1 and periodically in subsequent cycles. Blood and/or plasma samples may also be evaluated for the presence of metabolites of Compound 1 and to assess additional PK and PD endpoints. Efficacy will be assessed using standard Response Evaluation Criteria in Solid Tumors (RECIST) version (v) 1.1 criteria with assessments performed every 9 weeks (Q9W) for the first 27 weeks and every 12 weeks (Q12W) thereafter until documented disease progression. Tumor biomarkers will be collected, and documentation of progression will require radiologic evidence of progression or unequivocal demonstration of clinical progression.

What is claimed is:

1. A method of treating a cancer in a patient in need thereof, comprising administering to the patient (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof.

2. A method of treating a cancer in a patient in need thereof, comprising administering to the patient a pharmaceutical composition comprising (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof, and at least one pharmaceutically acceptable excipient.

3. The method of claim 1 or 2, wherein the cancer is breast cancer.

4. The method of claim 3, wherein the cancer is a hormone positive breast cancer.

5. The method of claim 3, wherein the cancer is ER positive breast cancer.

6. The method of claim 3, wherein the cancer is a HER2 negative breast cancer.

7. The method of claim 3, wherein the cancer is a HER2 positive breast cancer.

8. The method of claim 3, wherein the cancer is a triple negative breast cancer.

9. The method of claim 3, wherein the cancer is an invasive ductal carcinoma.

10. The method of claim 3, wherein the cancer is an invasive lobular carcinoma.

11. The method of claim 3, wherein the cancer is a medullary carcinoma.

12. The method of claim 3, wherein the cancer is a metaplastic carcinoma.

13. The method of claim 1 or 2, wherein the cancer is ovarian, fallopian tube, or primary peritoneal cancer.

14. The method of claim 13, wherein the cancer is a mucinous ovarian cancer.

15. The method of claim 13, wherein the cancer is a high-grade serous ovarian cancer.

16. The method of claim 13, wherein the cancer is a clear cell ovarian cancer.

17. The method of claim 13, wherein the cancer is an endometrioid ovarian cancer.

18. The method of claim 13, wherein the cancer is an epithelial ovarian cancer.

19. The method of claim 1 or 2, wherein the cancer is prostate cancer.

20. The method of claim 19, wherein the cancer is a metastatic castration-resistant prostate cancer (CRPC).

21. The method of claim 19, wherein the cancer is a metastatic castration-sensitive prostate cancer (CSPC).

22. The method of claim 19, wherein the cancer is high or very high-risk prostate cancer.

23. The method of claim 19, wherein the cancer is a biochemical recurrent prostate cancer with rising PSA.

24. The method of claim 1 or 2, wherein the cancer is gastric, esophageal, or gastro-esophageal junction (GEJ) cancer.

25. The method of claim 1 or 2, wherein the cancer is endometrial or uterine cancer.

26. The method of claim 25, wherein the cancer is a serous endometrial cancer.

27. The method of claim 25, wherein the cancer is an endometrioid endometrial cancer.

28. The method of claim 25, wherein the cancer is a microsatellite stable (MSS) endometrial cancer.

29. The method of claim 25, wherein the cancer is an endometrial cancer with microsatellite instability (MSI).

30. The method of claim 1 or 2, wherein the cancer is cervical cancer.

31. The method of claim 1 or 2, wherein the cancer is lung cancer.

32. The method of claim 25, wherein the cancer is an adenocarcinoma non-small cell lung cancer.

33. The method of claim 31, wherein the cancer is a squamous cell carcinoma non-small cell lung cancer.

34. The method of claim 31, wherein the cancer is a small cell lung cancer.

35. The method of claim 1 or 2, wherein the cancer is pancreatic cancer.

36. The method of claim 1 or 2, wherein the cancer is selected from colorectal, anal, or biliary tract cancer.

37. The method of claim 36, wherein the cancer is a microsatellite stable (MSS) colorectal cancer.

38. The method of claim 36, wherein the cancer is a colorectal cancer with microsatellite instability (MSI).

39. The method of claim 1 or 2, wherein the cancer is skin cancer.

40. The method of claim 39, wherein the cancer is melanoma.

41. The method of claim 1 or 2, wherein the cancer is a cancer associated with the CNS system.

42. The method of claim 41, wherein the cancer is glioblastoma.

43. The method of claim 1 or 2, wherein the cancer is a hematopoietic cancer.

44. The method of claim 43, wherein the cancer is acute myeloid leukemia (AML).

45. The method of claim 43, wherein the cancer is myelodysplastic syndrome (MDS).

46. The method of claim 43, wherein the cancer is diffuse large B cell lymphoma (DLBCL).

47. The method of claim 1 or 2, wherein the cancer is a head and neck cancer.

48. The method of claim 1 or 2, wherein the cancer is thyroid cancer.

49. The method of claim 1 or 2, wherein the cancer is a sarcoma.

50. The method of any one of claims 1-49, wherein the cancer is characterized as a having a deficiency in single stranded break repair (SSBR).

51. The method of any one of claims 1-49, wherein the cancer is characterized as a having a deficiency in base excision repair (BER).

52. The method of any one of claims 1-49, wherein the cancer is characterized as a having a deficiency in protection of the DNA replication fork.

53. The method of any one of claims 1-52, wherein the cancer exhibits a mis-regulation in expression of genes selected from BRCA2, BOD1L, DNA-PKC, FEN1, FBH1, POLB, LIG3, PARP1, PCNA, POLK, RAD51, RAD52, RECQ1, RPA, WRN, WRNIP1, XRCC1, or 53BP1.

54. The method of any one of claims 1-49, wherein the cancer is characterized as a homologous repair deficient (HRD) cancer.

55. The method of any one of claim 1-49, or 54, wherein the cancer exhibits a mis-regulation in expression of genes selected from BRCA1, BRCA2, RAD54, RAD51B, ATM, BARDI, CHK1, CHK2, CDK12, RAD51B, RAD54L, RAD51C, RAD51D, PPP22A, BRIPI, CTIP, PALB2, XRCC2, BLM, WRN, or NBS.

56. The method of any one of claim 1-49, or 54, wherein the cancer exhibits a mis-regulation in expression of genes encoding Fanconi anemia (FA) proteins.

57. The method of any one of claim 1-49, or 54, wherein the cancer exhibits a mis-regulation in expression of genes encoding FA-like genes selected from FANCA, FANCB, FANCC, FANCDI (BRCA2), FANCD2, FANCE, FANCE, FANCG, FANCI, FANJ (BRIPI), FANCL, FANCM, FANCN (RALB2), FANCP (SLX4), FANCS (BRCA1), RAD51C, or XPF.

58. The method of claim 54, wherein the cancer is characterized by loss of heterozygosity (LOH), telomeric allelic imbalance (TAI), large-scale state transitions (LST), epigenetic events, or BRCA promoter methylation.

59. The method of any one of claims 1-49, wherein the cancer is characterized as a having a deficiency in regulation of DNA damage response or cell cycle checkpoints.

60. The method of claim 59, wherein the cancer exhibits a mis-regulation in expression of genes associated with regulation of DNA damage response or cell cycle checkpoints selected from ATM, ATR, CHK1, CHK2, DNA-PK, PKMYT1, USP1, WEE1, DNA polymerase theta, CDK1, CDK2, CDK4, or CDK6.

61. The method of any one of claims 1-49, wherein the cancer is characterized as a having a mis-regulation in expression of a tumor suppressor gene.

62. The method of claim 61, wherein the tumor suppressor gene is selected from TP53 or PTEN1.

63. The method of any one of claims 1-49, wherein the cancer is characterized as a having a mis-regulation in an oncogene.

64. The method of claim 63, wherein the oncogene is selected from K-RAS, phosphatidylinositol 3-kinases (PI3K), or BRAF.

65. The method of any one of claims 1-49, wherein the cancer is characterized as having amplification of CCNE1.

66. The method of any one of the preceding claims, wherein the cancer is metastatic.

67. The method of any one of the preceding claims, wherein the method is adjuvant therapy following surgical resection.

68. The method of any one of the preceding claims, wherein the method is neo-adjuvant therapy.

69. The method of any one of claims 1-65, wherein the patient has relapsed after prior therapy.

70. The method of claim 69, wherein the patient has relapsed after treatment with a platinum-based chemotherapy.

71. The method of claim 69, wherein the patient has relapsed after treatment with a PARP inhibitor.

72. The method of any one of claims 1-65, wherein the patient has acquired resistance to prior therapy.

73. The method of claim 72, wherein the patient has acquired resistance to a platinum-based chemotherapy.

74. The method of claim 72, wherein the patient has acquired resistance to treatment with a PARP inhibitor.

75. The method of claim 72, wherein the patient has acquired resistance to treatment due to a BRCA1 or BRCA2 reversion mutation.

76. The method of any one of claims 1-65, wherein the patient is refractory to therapy.

77. The method of claim 76, wherein the patient is refractory to a platinum-based chemotherapy.

78. The method of claim 76, wherein the patient is refractory to treatment with a PARP inhibitor.

79. A method of treating a cancer in a patient in need thereof, comprising administering to the patient:
(a) a composition comprising (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof; and
(b) at least one oncology therapeutic selected from a DNA damaging agent, a platinum-based chemotherapy, a DNA damage response inhibitor, cell cycle checkpoint inhibitor, a nucleotide metabolism inhibitor, a nucleotide synthesis inhibitor, an endocrine therapy, a hormonal therapy, a SERM therapy, a SERD therapy, an androgen deprivation therapy, a novel hormone therapy, a taxane, a topoisomerase inhibitor, an anti-VEGF therapeutic, an immune checkpoint inhibitor, a kinase inhibitor, a K-RAS inhibitor, a phosphatidylinositol 3-kinases (PI3K) inhibitor, an antibody drug conjugate (ADC), or a radiopharmaceutical.

80. The method of claim 79, wherein at least one oncology therapeutic is a DNA damaging agent.

81. The method of claim 80, wherein the DNA damaging agent is selected from hydroxyurea or temozolomide.

82. The method of claim 79, wherein at least one oncology therapeutic is a platinum-based chemotherapy.

83. The method of claim 81, wherein the platinum-based chemotherapy is selected from cisplatin, carboplatin, satraplatin, heptaplatin, picoplatin, nedaplatin, triplatin, lipoplatin, or oxaliplatin.

84. The method of claim 79, wherein at least one oncology therapeutic is a DNA damage response inhibitor or a cell cycle checkpoint inhibitor.

85. The method of claim 84, wherein the DNA damage response inhibitor or cell cycle checkpoint inhibitor is selected from an ATM, ATR, CHK1, CHK2, DNA-PK, PKMYT1, USP1, WEE1, DNA polymerase theta, CDK1, CDK2, CDK4, or CDK6 inhibitor.

86. The method of claim 84, wherein the DNA damage response inhibitor or cell cycle checkpoint inhibitor is selected from a CDK4 and CDK6 dual inhibitor.

87. The method of claim 84, wherein the DNA damage response inhibitor or cell cycle checkpoint inhibitor is selected from a KAT6A, KAT6B, or KAT6A/B inhibitor.

88. The method of claim 84, wherein the DNA damage response inhibitor or cell cycle checkpoint inhibitor is a kinase inhibitor.

89. The method of claim 85 or 88, wherein the DNA damage response inhibitor or cell cycle checkpoint inhibitor is selected from KU-60019, KU-55933, CP-466722, AZD0156, AZD1390, M3541, M4076, XRD-0394, berzosertib, camonsertib, ceralasertib, elimusertib, gartisertib, tuvusertib, IMP9064, ATG-018, ATRN-119, ART0380, dactolisib, rabusertib, prexasertib, AZD7762, BML-277, CHIR-124, CCT245737, GDC-0575, MK-8776, PD0166285, PF-477736, nedisertib, samotolisib, peposertib, AZD7648, CC-115, BR2002, BR101801, LTURM34, LY293646, NU7441, RP-6306, GSK-1520489A, KSQ-4279, ML323, TNG348, ISM3091, adavosertib, azenosertib, Debio0123, IMP7068, PD0166285, SY-4835, ACR-368, ACR-2316, ART558, ART812, ART4215, RP-6685, IDE705, abemaciclib, palbociclib, ribociclib, PF-07104091, BLU-222, INX-315, or ARTS-021.

90. The method of claim 85 or 88, wherein the DNA damage response inhibitor or cell cycle checkpoint inhibitor is selected from PF-07248144, OP-3136, or PF-07220060.

91. The method of claim 79, wherein at least one oncology therapeutic is a nucleotide metabolism inhibitor or a nucleotide synthesis inhibitor.

92. The method of claim 91, wherein the nucleotide metabolism inhibitor or nucleotide synthesis inhibitor is selected from 5-fluorouracil, pemetrexed, leflunomide, brequinar, or methotrexate.

93. The method of claim 79, wherein at least one oncology therapeutic is an endocrine therapy, a hormonal therapy, a SERM therapy, or a SERD therapy.

94. The method of claim 93, wherein the endocrine therapy, hormonal therapy, SERM therapy, or SERD therapy is selected from tamoxifen, torimifene, anastrozole, letrozole, exemestane, buserelin, goserelin, leuprorelin, triptorelin, fulvestrant, camizestrant, elacestrant, amcenestrant, giredestrant, imlunestrant, rintodestrant, SHR9549, ZN-c5, D0502, vepdegrestrant, palazestrant, AC682, DT2216, or degarelix.

95. The method of claim 79, wherein at least one oncology therapeutic is an androgen deprivation therapy or a novel hormone therapy.

96. The method of claim 95, wherein the androgen deprivation therapy or novel hormone therapy is selected from abiraterone, orteronel, bicalutamide, flutamide, enzalutamide, apalutamide, darolutamide, nilutamide, ODM201, AZD3514, BMS641988, ARV-110, ARV-766, CC-94676, AC-0176, HP518, GT20029, or cyproterone acetate.

97. The method of claim 79, wherein at least one oncology therapeutic is a taxane.

98. The method of claim 97, wherein the taxane is selected from paclitaxel, docetaxel, cabazitaxel, or abraxane.

99. The method of claim 79, wherein at least one oncology therapeutic is a topoisomerase inhibitor.

100. The method of claim 99, wherein the topoisomerase inhibitor is selected from irinotecan, topotecan, belotecan, doxorubicin, epirubicin, idarubicin, camptothecin, or etoposide.

101. The method of claim 79, wherein at least one oncology therapeutic is an anti-VEGF therapeutic.

102. The method of claim 101, wherein the anti-VEGF therapeutic is selected from bevacizumab, ramucirumab, aflibercept, sunitinib, pazopanib, regorafenib, or lenvatinib.

103. The method of claim 79, wherein at least one oncology therapeutic is an immune checkpoint inhibitor.

104. The method of claim 103, wherein the immune checkpoint inhibitor is selected from a CTLA-4 inhibitor, a PD-1 inhibitor, a PD-L1 inhibitor, or a bi-specific PD-1/CTLA4 inhibitor.

105. The method of claim 104, wherein the CTLA-4 inhibitor is selected from ipilimumab or tremelimumab.

106. The method of claim 104, wherein the PD-1 inhibitor is selected from spartalizumab, nivolumab, pembrolizumab, cemiplimab, atezolizumab, avelumab, durvalumab, dostarlimab, retifanlimab, or toripalimab.

107. The method of claim 104, wherein the bi-specific PD-1/CTLA4 inhibitor is selected from AK104, MGD019, XmAb20717, or MEDI5752.

108. The method of claim 103, wherein the immune checkpoint inhibitor is selected from a bi-specific PD-1/VEGF inhibitor.

109. The method of claim 108, wherein the bi-specific PD-1/VEGF inhibitor is selected from ivonescimab, LM-299, IMM2510, JS207, AK112, or AI-081.

110. The method of claim 79, wherein at least one oncology therapeutic is a kinase inhibitor.

111. The method of claim 79, wherein at least one oncology therapeutic is a K-RAS inhibitor.

112. The method of claim 111, wherein the K-RAS inhibitor is selected from sotorasib, adagrasib, opnurasib, or garsorasib.

113. The method of claim 111, wherein the K-RAS inhibitor is selected from olomorasib.

114. The method of claim 79, wherein at least one oncology therapeutic is a phosphoinositide 3-kinase inhibitor.

115. The method of claim 114, wherein the phosphoinositide 3-kinase inhibitor is selected from copanlisib, alpelisib, idelalisib, duvelisib, or umbralisib.

116. The method of claim 79, wherein at least one oncology therapeutic is a BRAF inhibitor.

117. The method of claim 116, wherein the BRAF inhibitor is selected from vemurafenib, dabrafenib or encorafenib.

118. The method of claim 79, wherein at least one oncology therapeutic is an AKT inhibitor.

119. The method of claim 118, wherein the AKT inhibitor is capivasertib.

120. The method of claim 79, wherein at least one oncology therapeutic is an antibody drug conjugate.

121. The method of claim 120, wherein the antibody drug conjugate is selected from ado-trastuzumab emtansine, enfortumab vedotin, fam-trastuzumab deruxtecan-nxki, sacituzumab govitecan, cetuximab sarotalocan, disitamab vedotin, tisotumab vedotin, datopotamab deruxtecan, MK-2870, raludotatug deruxtecan, ifinatamab deruxtecan, HS-20089, patritumab deruxtecan, mirvetuximab soravtansine, or farletuzumab ecteribulin.

122. The method of claim 106, wherein the antibody drug conjugate is selected from rinatabart sesutecan, telisotuzumab adizutecan, ABBV-706, YL201, AZD8205, SGN-B7H4V, GSK5764227, or DS-7300.

123. The method of claim 79, wherein at least one oncology therapeutic is a radiopharmaceutical.

124. The method of claim 123, wherein the radiopharmaceutical is selected from lutetium Lu 177 vipivotide tetraxetan, radium-223 dichloride, sodium iodide I-131, lobenguane iodine-131, lutetium-177, or yttrium-90.

125. The method of any one of the preceding claims, wherein the (R)-1-methyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide, or pharmaceutically acceptable salt or solvate thereof, is administered orally.

126. The method of any one of the preceding claims, wherein the oral administration occurs once per day, twice per day, three times per day, every other day, or one to five days per week.

127. The method of any one of the preceding claims, wherein the method further comprises administration to the patient of a NAD+ precursor.

128. The method of claim 127 wherein the NAD+ precursor is selected from nicotinic acid, nicotinamide, nicotinamide riboside, or dihydronicotinamide riboside.
